(19) United States
(12) Patent Application Publication
(10) Pub. No.: US 2023/0355332 A1
(43) Pub. Date: Nov. 9, 2023

(54) MEDICAL ARM CONTROL SYSTEM, MEDICAL ARM DEVICE, MEDICAL ARM CONTROL METHOD, AND PROGRAM

(71) Applicant: SONY GROUP CORPORATION, TOKYO (JP)

(72) Inventor: DAISUKE NAGAO, TOKYO (JP)

(21) Appl. No.: 18/043,623

(22) PCT Filed: Aug. 16, 2021
(86) PCT No.: PCT/JP2021/029898

§ 371 Date: Mar. 1, 2023

(30) Foreign Application Priority Data
Sep. 11, 2020 (JP) 2020-152575

(51) Int. Cl. A61B 34/32; A61B 34/00; B25J 9/16

(52) U.S. Cl. CPC A61B 34/32; A61B 34/70; B25J 9/161; B25J 9/163; A61B 2034/301

(57) ABSTRACT

Provided is a medical arm control system including a first determination unit (222) that performs supervised learning using first input data and first training data and generates an autonomous movement control model for autonomously moving a medical arm, a second determination unit (224) that performs supervised learning using second input data and second training data and generates a reward model for calculating a reward to be given to a movement of the medical arm, and a reinforcement learning unit (230) that executes the reward model using third input data and reinforces the autonomous movement control model using the reward calculated by the reward model.

### FIELD

The present disclosure relates to a medical arm control system, a medical arm device, a medical arm control method, and a program.

### BACKGROUND

In recent years, in endoscopic surgery, surgery is performed while capturing an image of an abdominal cavity of a patient using an endoscope and displaying the image captured by the endoscope on a display. For example, Patent Literature 1 below discloses a technique for interlocking control of an arm supporting the endoscope with control of electronic zoom of the endoscope.

### CITATION LIST

### Patent Literature

Patent Literature 1: WO 2018/159328 A

### SUMMARY

### Technical Problem

In recent years, development for autonomously moving a robot arm device that supports an endoscope has been advanced. For example, a learning device is caused to perform machine learning of surgery content and information associated with movement of a surgeon and a scopist corresponding to the surgery content, thereby generating a learning model. Then, control information for autonomously controlling the robot arm device is generated with reference to the learning model, the control rule, and the like obtained in this manner.

However, performance of movement of the robot arm device depends on human sensitivity, and thus it is difficult to model an ideal movement of the robot arm device. Therefore, it is conceivable to obtain a large amount of information (clinical data) regarding the movement of the robot arm device and perform machine learning of the information in order to acquire an ideal model for the movement of the robot arm device. However, since it is difficult to collect a large amount of information regarding the movement in a clinical field, it is difficult to efficiently construct a movement model supporting a wider range of situations.

Therefore, the present disclosure proposes a medical arm control system, a medical arm device, a medical arm control method, and a program capable of efficiently acquiring a learning model for autonomous movement in consideration of human sensitivity while covering a wider range of situations even in a case where only a small amount of clinical data can be obtained.

### Solution to Problem

According to the present disclosure, there is provided a medical arm control system including: a first determination unit that performs supervised learning using first input data and first training data, and generates an autonomous movement control model for autonomously moving a medical arm; a second determination unit that performs supervised learning using second input data and second training data, and generates a reward model for calculating a reward to be given to a movement of the medical arm; and a reinforcement learning unit that executes the reward model using third input data, and reinforces the autonomous movement control model using the reward calculated by the reward model.

Furthermore, according to the present disclosure, there is provided a medical arm device which stores an autonomous movement control model obtained by reinforcing a control model for autonomously moving a medical arm using a reward obtained by inputting third input data to a reward model for calculating the reward to be given to a movement of the medical arm, the control model being generated by performing supervised learning using first input data and first training data, the reward model being generated by performing supervised learning using second input data and second training data.

Furthermore, according to the present disclosure, there is provided a medical arm control method, by a medical arm control system, including: reinforcing an autonomous movement control model for autonomously moving the medical arm, using a reward obtained by inputting third input data to a reward model for calculating the reward to be given to a movement of the medical arm, the autonomous movement control model being generated by performing supervised learning using first input data and first training data, the reward model being generated by performing supervised learning using second input data and second training data; and controlling the medical arm using the reinforced autonomous movement control model.

Moreover, according to the present disclosure, there is provided a program causes a computer to function as a first determination unit that performs supervised learning using first input data and first training data, and generates an autonomous movement control model for autonomously moving a medical arm; a second determination unit that performs supervised learning using second input data and second training data, and generates a reward model for calculating a reward to be given to a movement of the medical arm; and a reinforcement learning unit that executes the reward model using third input data, and reinforces the autonomous movement control model using the reward calculated by the reward model.

### BRIEF DESCRIPTION OF DRAWINGS

FIG. 1 is a diagram illustrating an example of a schematic configuration of an endoscopic surgery system to which the technology according to the present disclosure can be applied.

FIG. 2 is a block diagram illustrating an example of a functional configuration of a camera head and a camera control unit (CCU) illustrated in FIG. 1.

FIG. 3 is a schematic diagram illustrating a configuration of a forward-oblique viewing endoscope according to an embodiment of the present disclosure.

FIG. 4 is a diagram illustrating an example of a configuration of a medical observation system 10 according to the embodiment of the present disclosure.

FIG. 5 is a block diagram illustrating a configuration example of a learning device 200 according to the embodiment of the present disclosure.

FIG. 6 is a flowchart illustrating an example of a model generation method according to the embodiment of the present disclosure.

FIG. 7 is an explanatory diagram illustrating an example of a method for generating an autonomous movement control model according to the embodiment of the present disclosure.

FIG. 8 is an explanatory diagram illustrating an example of a method for generating a reward model according to the embodiment of the present disclosure.

FIG. 9 is a flowchart illustrating an example of reinforcement learning according to the embodiment of the present disclosure.

FIG. 10 is an explanatory diagram illustrating an example of reinforcement learning according to the embodiment of the present disclosure.

FIG. 11 is a block diagram illustrating an example of a configuration of a control device 300 according to the embodiment of the present disclosure.

FIG. 12 is a flowchart illustrating an example of a control method according to the embodiment of the present disclosure.

FIG. 13 is an explanatory diagram illustrating the control method according to the embodiment of the present disclosure.

FIG. 14 is a hardware configuration diagram illustrating an example of a computer that implements the learning device 200 according to the embodiment of the present disclosure.

### DESCRIPTION OF EMBODIMENTS

Hereinafter, preferred embodiments of the present disclosure will be described in detail with reference to the accompanying drawings. Note that, in the present specification and the drawings, components having substantially the same functional configuration are denoted by the same reference signs to omit redundant description. In addition, in the present specification and the drawings, a plurality of components having substantially the same functional configurations may be distinguished by attaching a different alphabet after the same reference sign. However, when it is not particularly necessary to distinguish each of the plurality of components having substantially the same or similar functional configuration, only the same reference sign is assigned.

The description will be given in the following order.
1. Configuration example of endoscopic surgery system 5000
1.1 Schematic configuration of endoscopic surgery system 5000
1.2 Detailed configuration example of support arm device 5027
1.3 Detailed configuration example of light source device 5043
1.4 Detailed configuration example of camera head 5005 and CCU 5039
1.5 Configuration example of endoscope 5001
2. Configuration example of medical observation system 10
3. Background to creation of embodiment of present disclosure
4. Embodiment
4.1 Detailed configuration of learning device 200
4.2 Method for generating autonomous movement control model
4.3 Method for generating reward model
4.4 Method for reinforcing autonomous movement control model
4.5 Detailed configuration of control device 300
4.6 Control method
5. Summary
6. Hardware configuration
7. Supplement

### <<1. Configuration Example of Endoscopic Surgery System 5000>>

### <1.1 Schematic Configuration of Endoscopic Surgery System 5000>

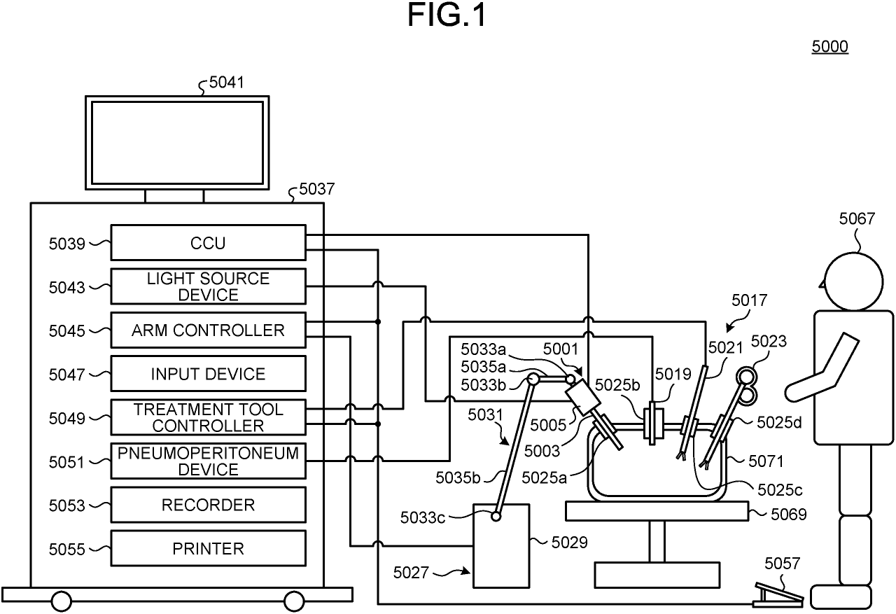

First, before describing details of an embodiment of the present disclosure, a schematic configuration of an endoscopic surgery system 5000 to which a technology according to the present disclosure can be applied will be described with reference to FIG. 1. FIG. 1 is a diagram illustrating an example of the schematic configuration of the endoscopic surgery system 5000 to which the technology according to the present disclosure can be applied. FIG. 1 illustrates a situation in which a surgeon 5067 is performing surgery on a patient 5071 on a patient bed 5069, using the endoscopic surgery system 5000. As illustrated in FIG. 1, the endoscopic surgery system 5000 includes an endoscope 5001, other surgical instruments (medical instruments) 5017, a support arm device (medical arm) 5027 that supports the endoscope (medical observation device) 5001, and a cart 5037 on which various devices for endoscopic surgery are mounted. Hereinafter, details of the endoscopic surgery system 5000 will be sequentially described.

### (Surgical Instruments 5017)

In the endoscopic surgery, instead of making an incision in the abdominal wall to open the abdomen, a plurality of tube-like puncture instruments called trocars 5025a to 5025d is punctured into the abdominal wall. Then, a lens barrel 5003 of the endoscope 5001 and other surgical instruments 5017 are inserted into a body cavity of the patient 5071 from the trocars 5025a to 5025d. In the example illustrated in FIG. 1, as other surgical instruments 5017, a pneumoperitoneum tube 5019, an energy treatment tool 5021, and forceps 5023 are inserted into the body cavity of the patient 5071. The energy treatment tool 5021 is a treatment tool that performs incision and ablation of tissue, sealing of a blood vessel, or the like by high-frequency current or ultrasonic vibration. However, the surgical instruments 5017 illustrated in FIG. 1 are merely examples, and the surgical instruments 5017 may include various surgical instruments generally used in the endoscopic surgery, such as tweezers and a retractor.

### (Support Arm Device 5027)

The support arm device 5027 includes an arm 5031 extending from a base 5029. In the example illustrated in FIG. 1, the arm 5031 includes joints 5033a, 5033b, and 5033c and links 5035a and 5035b, and is driven by control of an arm controller 5045. The arm 5031 supports the endoscope 5001 to control a position and an attitude of the endoscope 5001. As a result, the endoscope 5001 can be stably fixed in the position.

### (Endoscope 5001)

The endoscope 5001 includes a lens barrel 5003 whose region of a predetermined length from a distal end is inserted into the body cavity of the patient 5071, and a camera head 5005 connected to a proximal end of the lens barrel 5003. In the example in FIG. 1, the endoscope 5001 configured as a so-called rigid scope having a rigid lens barrel 5003 is illustrated, but the endoscope 5001 may be configured as a so-called flexible scope having a flexible lens barrel 5003. The embodiment of the present disclosure is not particularly limited.

An opening into which an objective lens is fitted is provided at a distal end of the lens barrel 5003. A light source device 5043 is connected to the endoscope 5001, and light generated by the light source device 5043 is guided to the distal end of the lens barrel by a light guide extending inside the lens barrel 5003, and is emitted toward an observation target in the body cavity of the patient 5071 via the objective lens. Note that, in the embodiment of the present disclosure, the endoscope 5001 may be a forward-viewing endoscope or a forward-oblique viewing endoscope, and is not particularly limited.

An optical system and an imaging element are provided inside the camera head 5005, and reflected light (observation light) from the observation target is condensed on the imaging element by the optical system. The observation light is photoelectrically converted by the imaging element, and an electric signal corresponding to the observation light, i.e., an image signal corresponding to the observation image, is generated. The image signal is transmitted to a camera control unit (CCU) 5039 as RAW data. Note that the camera head 5005 has a function of adjusting a magnification and a focal length by appropriately driving the optical system.

For example, in order to support a stereoscopic vision (3D display) or the like, a plurality of imaging elements may be provided in the camera head 5005. In this case, a plurality of relay optical systems will be provided inside the lens barrel 5003 in order to guide the observation light to each of the plurality of imaging elements.

### (Various Devices Mounted on Cart)

First, a display device 5041 displays an image based on the image signal subjected to image processing by the CCU 5039 under the control of the CCU 5039. In a case where the endoscope 5001 supports high-resolution imaging such as 4 K (number of horizontal pixels 3840 × number of vertical pixels 2160) or 8 K (number of horizontal pixels 7680 × number of vertical pixels 4320), and/or in a case where the endoscope supports the 3D display, a display device capable of a high-resolution display and/or a display device capable of the 3D display is used as the display device 5041. Furthermore, a plurality of display devices 5041 having different resolutions and sizes may be provided depending on application.

Furthermore, an image of a surgical site in the body cavity of the patient 5071 captured by the endoscope 5001 is displayed on the display device 5041. While viewing the image of the surgical site displayed on the display device 5041 in real time, the surgeon 5067 can perform treatment such as resection of an affected part using the energy treatment tool 5021 and the forceps 5023. Although not illustrated, the pneumoperitoneum tube 5019, the energy treatment tool 5021, and the forceps 5023 may be supported by the surgeon 5067, an assistant, or the like during surgery.

Furthermore, the CCU 5039 includes a central processing unit (CPU), a graphics processing unit (GPU), and the like, and can integrally control movement of the endoscope 5001 and the display device 5041. Specifically, the CCU 5039 performs, on the image signal received from the camera head 5005, various types of image processing for displaying an image based on the image signal, such as development processing (demosaic processing). Further, the CCU 5039 provides the image signal subjected to the image processing to the display device 5041. Furthermore, the CCU 5039 transmits a control signal to the camera head 5005 and controls driving thereof. The control signal may include information regarding imaging conditions such as the magnification and the focal length.

The light source device 5043 includes a light source such as a light emitting diode (LED), and supplies irradiation light for photographing the surgical site to the endoscope 5001.

The arm controller 5045 includes, for example, a processor such as a CPU, and operates according to a predetermined program to control driving of the arm 5031 of the support arm device 5027 according to a predetermined control system.

An input device 5047 is an input interface for the endoscopic surgery system 5000. The surgeon 5067 can input various types of information and instructions to the endoscopic surgery system 5000 via the input device 5047. For example, the surgeon 5067 inputs various types of information regarding surgery, such as physical information of a patient and information regarding a surgical procedure of the surgery, via the input device 5047. Furthermore, for example, the surgeon 5067 can input an instruction to drive the arm 5031, an instruction to change imaging conditions (type, magnification, focal length, and the like of irradiation light) by the endoscope 5001, an instruction to drive the energy treatment tool 5021, and the like via the input device 5047. Note that the type of the input device 5047 is not limited, and the input device 5047 may be various known input devices. As the input device 5047, for example, a mouse, a keyboard, a touch panel, a switch, a foot switch 5057, and/or a lever may be applied. For example, when the touch panel is used as the input device 5047, the touch panel may be provided on a display surface of the display device 5041.

Alternatively, the input device 5047 may be a device worn on a part of the body of the surgeon 5067, such as an eyeglass shaped wearable device or a head mounted display (HMD). In this case, various inputs are performed according to gesture or a line of sight of the surgeon 5067 detected by these devices. Furthermore, the input device 5047 can include a camera capable of detecting movement of the surgeon 5067, and various inputs may be performed according to a gesture or a line of sight of the surgeon 5067 detected from an image captured by the camera. Furthermore, the input device 5047 can include a microphone capable of collecting voice of the surgeon 5067, and various inputs may be performed by voice via the microphone. As described above, the input device 5047 is configured to be able to input various types of information in a non-contact manner, and thus, in particular, a user (e.g., surgeon 5067) in a clean area can operate a device in an unclean area in a non-contact manner. In addition, since the surgeon 5067 can operate the instrument without releasing his/her hand from the surgical instrument held, convenience of the surgeon 5067 is improved.

A treatment tool controller 5049 controls driving of the energy treatment tool 5021 for cauterization of tissue, incision, sealing of a blood vessel, or the like. A pneumoperitoneum device 5051 feeds gas into the body cavity of the patient 5071 via the pneumoperitoneum tube 5019 in order to inflate the body cavity for the purpose of securing a visual field of the endoscope 5001 and securing a work space for the surgeon 5067. A recorder 5053 is a device capable of recording various types of information regarding surgery. A printer 5055 is a device capable of printing various types of information regarding surgery in various formats such as text, image, or graph.

### <1.2 Detailed Configuration Example of Support Arm Device 5027>

Furthermore, an example of a detailed configuration of the support arm device 5027 will be described. The support arm device 5027 includes a base 5029 that is a base and an arm 5031 extending from the base 5029. In the example illustrated in FIG. 1, the arm 5031 includes a plurality of joints 5033a, 5033b, and 5033c and a plurality of links 5035a and 5035b connected by the joint 5033b, but in FIG. 1, the configuration of the arm 5031 is illustrated in a simplified manner for the sake of simplicity. Specifically, a shape, number, and arrangement of the joints 5033a to 5033c and the links 5035a and 5035b, a direction of rotation axes of the joints 5033a to 5033c, and the like can be appropriately set so that the arm 5031 has a desired degree of freedom. For example, the arm 5031 can be suitably configured to have six degrees of freedom or more. As a result, since the endoscope 5001 can be freely moved within a movable range of the arm 5031, the lens barrel 5003 of the endoscope 5001 can be inserted into the body cavity of the patient 5071 from a desired direction.

Actuators are provided in the joints 5033a to 5033c, and the joints 5033a to 5033c are configured to be rotatable around predetermined rotation axes by driving the actuators. The arm controller 5045 controls the driving of the actuators, so that a rotation angle of each of the joints 5033a to 5033c is controlled to drive the arm 5031. As a result, the position and the attitude of the endoscope 5001 are controlled. At this time, the arm controller 5045 can control driving of the arm 5031 by various known control systems such as force control or position control.

For example, when the surgeon 5067 appropriately performs an operation input via the input device 5047 (including the foot switch 5057), the driving of the arm 5031 is appropriately controlled by the arm controller 5045 according to the operation input, and the position and attitude of the endoscope 5001 may be controlled. Note that the arm 5031 may be manipulated by a so-called primary/replica (master slave) system. In this case, the arm 5031 (arm included in a patient-side cart) may be remotely manipulated by the surgeon 5067 via the input device 5047 (surgeon console) installed at a location remote from an operating room or within the operating room.

Here, in general, in the endoscopic surgery, the endoscope 5001 is supported by a doctor called a scopist. On the other hand, in the embodiment of the present disclosure, since the position of the endoscope 5001 can be more reliably fixed without manual support by using the support arm device 5027, an image of the surgical site can be stably obtained, and the surgery can be smoothly performed.

Note that the arm controller 5045 is not necessarily provided in the cart 5037. Furthermore, the arm controller 5045 is not necessarily a single device. For example, the arm controller 5045 may be provided in each of the joints 5033a to 5033c of the arm 5031 of the support arm device 5027, and the drive control of the arm 5031 may be realized by a plurality of arm controllers 5045 cooperating with each other.

### <1.3 Detailed Configuration Example of Light Source Device 5043>

Next, an example of a detailed configuration of the light source device 5043 will be described. The light source device 5043 supplies the endoscope 5001 with irradiation light for capturing an image of the surgical site. The light source device 5043 is configured with a white light source of, for example, an LED, a laser light source, or a combination thereof. Here, when the white light source is configured with a combination of RGB laser light sources, an output intensity and an output timing of each color (each wavelength) can be controlled with high accuracy, so that a white balance of a captured image can be adjusted in the light source device 5043. Furthermore, in this case, by irradiating the observation target with the laser light from each of the RGB laser light sources in a time division manner and controlling the driving of the imaging element of the camera head 5005 in synchronization with the irradiation timing, it is also possible to capture an image corresponding to each of RGB in a time division manner. According to this method, a color image can be obtained without providing a color filter in the imaging element.

Furthermore, the driving of the light source device 5043 may be controlled so as to change an intensity of light to be output every predetermined time. By controlling the driving of the imaging element of the camera head 5005 in synchronization with the timing of the change of the light intensity to acquire images in a time division manner and synthesizing the images, it is possible to generate an image of a high dynamic range without so-called blocked up shadows and blown out highlights.

Furthermore, the light source device 5043 may be configured to be able to supply light in a predetermined wavelength band corresponding to special light observation. In the special light observation, for example, light in a narrower band than the irradiation light (i.e., white light) for normal observation is irradiated to perform so-called narrow band imaging in which a predetermined tissue such as a blood vessel in a mucosal surface layer is imaged with high contrast by utilizing wavelength dependency of light absorption in a body tissue. Alternatively, in the special light observation, fluorescence observation for obtaining an image by fluorescence generated by irradiation with excitation light may be performed. In the fluorescence observation, for example, fluorescence from a body tissue can be observed by irradiating the body tissue with excitation light (autofluorescence observation), or a fluorescent image can be obtained by locally injecting a reagent such as indocyanine green (ICG) into the body tissue and irradiating the body tissue with excitation light corresponding to a fluorescence wavelength of the reagent. The light source device 5043 may be configured to supply narrow band light and/or excitation light corresponding to the special light observation.

### <1.4 Detailed Configuration Example of Camera Head 5005 and CCU 5039>

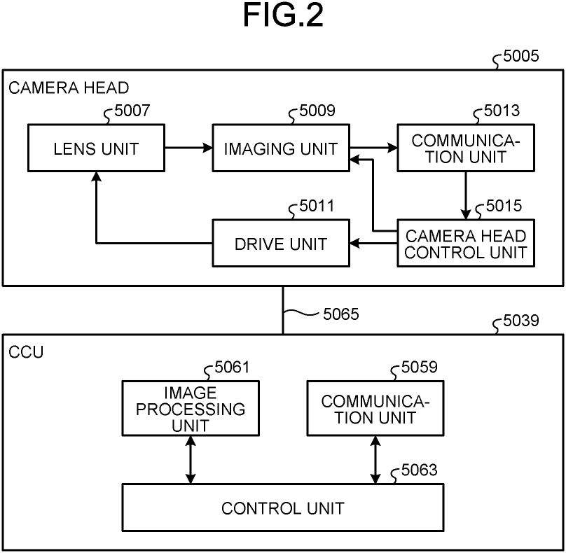

Next, an example of a detailed configuration of the camera head 5005 and the CCU 5039 will be described with reference to FIG. 2. FIG. 2 is a block diagram illustrating an example of functional configurations of the camera head 5005 and the CCU 5039 illustrated in FIG. 1.

Specifically, as illustrated in FIG. 2, the camera head 5005 includes, as functions thereof, a lens unit 5007, an imaging unit 5009, a drive unit 5011, a communication unit 5013, and a camera head control unit 5015. Furthermore, the CCU 5039 includes, as functions thereof, a communication unit 5059, an image processing unit 5061, and a control unit 5063. Then, the camera head 5005 and the CCU 5039 are connected to be bidirectionally communicable by a transmission cable 5065.

First, a functional configuration of the camera head 5005 will be described. The lens unit 5007 is an optical system provided in a connected part with the lens barrel 5003. Observation light taken in from the distal end of the lens barrel 5003 is guided to the camera head 5005 and enters the lens unit 5007. The lens unit 5007 is configured by combining a plurality of lenses including a zoom lens and a focus lens. Optical characteristics of the lens unit 5007 are adjusted so as to condense the observation light on a light receiving surface of the imaging element of the imaging unit 5009. In addition, the zoom lens and the focus lens are configured such that their positions on the optical axis are movable in order to adjust the magnification and the focal point of a captured image.

The imaging unit 5009 includes an imaging element and is arranged at a subsequent stage of the lens unit 5007. The observation light passing through the lens unit 5007 is condensed on the light receiving surface of the imaging element, and an image signal corresponding to the observation image is generated by photoelectric conversion. The image signal generated by the imaging unit 5009 is provided to the communication unit 5013.

As the imaging element configuring the imaging unit 5009, for example, a complementary metal oxide semiconductor (CMOS) type image sensor having a Bayer array for color imaging is used. Note that, as the imaging element, for example, an imaging element that can support capturing of a high-resolution image of 4K or more may be used. By obtaining the surgical site image with high resolution, the surgeon 5067 can grasp a state of the surgical site in more detail, and can thus perform the surgery more smoothly.

Furthermore, the imaging element configuring the imaging unit 5009 may be configured to include a pair of imaging elements for acquiring right-eye and left-eye image signals corresponding to 3D display (stereo system). By performing the 3D display, the surgeon 5067 can more accurately grasp a depth of a living tissue (organ) in the surgical site and grasp a distance to the living tissue. Note that, when the imaging unit 5009 is configured as a multiplate type, a plurality of lens units 5007 may be provided corresponding to the respective imaging elements.

Furthermore, the imaging unit 5009 is not necessarily provided in the camera head 5005. For example, the imaging unit 5009 may be provided immediately after the objective lens inside the lens barrel 5003.

The drive unit 5011 includes an actuator, and moves the zoom lens and the focus lens of the lens unit 5007 for a predetermined distance along the optical axis under the control of the camera head control unit 5015. As a result, the magnification and the focal point of the image captured by the imaging unit 5009 can be appropriately adjusted.

The communication unit 5013 includes a communication device for transmitting and receiving various types of information to and from the CCU 5039. The communication unit 5013 transmits the image signal obtained from the imaging unit 5009 as RAW data to the CCU 5039 via the transmission cable 5065. At this time, in order to display the captured image of the surgical site with low latency, the image signal is preferably transmitted by optical communication. This is because, at the time of surgery, the surgeon 5067 performs surgery while observing the state of an affected part using the captured image. For safer and more reliable surgery, it is required to display a moving image of the surgical site in real time as much as possible. In a case where optical communication is performed, the communication unit 5013 is provided with a photoelectric conversion module that converts an electric signal into an optical signal. The image signal is converted into an optical signal by the photoelectric conversion module and then transmitted to the CCU 5039 via the transmission cable 5065.

Furthermore, the communication unit 5013 receives a control signal for controlling driving of the camera head 5005 from the CCU 5039. The control signal includes, for example, information regarding imaging conditions such as information for specifying a frame rate of a captured image, information for specifying an exposure value at the time of imaging, and/or information for specifying the magnification and the focal point of the captured image. The communication unit 5013 provides the received control signal to the camera head control unit 5015. Note that the control signal from the CCU 5039 may also be transmitted by optical communication. In this case, the communication unit 5013 is provided with the photoelectric conversion module that converts the optical signal into the electric signal, and the control signal is converted into the electric signal by the photoelectric conversion module and then provided to the camera head control unit 5015.

Note that the imaging conditions such as the frame rate, the exposure value, the magnification, and the focal point are automatically set by the control unit 5063 of the CCU 5039 based on the acquired image signal. In other words, the endoscope 5001 has a so-called auto exposure (AE) function, an auto focus (AF) function, and an auto white balance (AWB) function.

The camera head control unit 5015 controls the driving of the camera head 5005 based on the control signal from the CCU 5039 received via the communication unit 5013. For example, the camera head control unit 5015 controls driving of the imaging element of the imaging unit 5009 based on the information to designate the frame rate of the captured image and/or the information for specifying an exposure at the time of imaging. Furthermore, for example, the camera head control unit 5015 appropriately moves the zoom lens and the focus lens of the lens unit 5007 via the drive unit 5011 based on the information to designate the magnification and the focal point of the captured image. The camera head control unit 5015 may further have a function of storing information for identifying the lens barrel 5003 and the camera head 5005.

Note that the camera head 5005 can have resistance to an autoclave sterilization process by arranging the lens unit 5007, the imaging unit 5009, and the like in a sealed structure having high airtightness and waterproofness.

Next, a functional configuration of the CCU 5039 will be described. The communication unit 5059 includes a communication device for transmitting and receiving various types of information to and from the camera head 5005. The communication unit 5059 receives the image signal transmitted from the camera head 5005 via the transmission cable 5065. At this time, as described above, the image signal can be suitably transmitted by optical communication. In this case, for the optical communication, the communication unit 5059 is provided with the photoelectric conversion module that converts the optical signal into the electrical signal. The communication unit 5059 provides the image signal converted into the electric signal to the image processing unit 5061.

Furthermore, the communication unit 5059 transmits the control signal for controlling the driving of the camera head 5005 to the camera head 5005. The control signal may also be transmitted by optical communication.

The image processing unit 5061 performs various types of image processing on the image signal that is RAW data transmitted from the camera head 5005. Examples of the image processing are various known signal processing including development processing, high image quality processing (band emphasis processing, super-resolution processing, noise reduction (NR) processing, and/or camera shake correction processing), and/or enlargement processing (electronic zoom processing). Furthermore, the image processing unit 5061 performs detection processing on the image signal for performing AE, AF, and AWB.

The image processing unit 5061 includes a processor such as a CPU or a GPU, and the processor operates according to a predetermined program to perform the above-described image processing and detection processing. Note that, when the image processing unit 5061 includes a plurality of GPUs, the image processing unit 5061 appropriately divides information related to the image signal, and performs image processing in parallel by the plurality of GPUs.

The control unit 5063 performs various types of control related to imaging of the surgical site by the endoscope 5001 and display of the captured image. For example, the control unit 5063 generates the control signal for controlling the driving of the camera head 5005. At this point, when imaging conditions are input by the surgeon 5067, the control unit 5063 generates the control signal based on the input by the surgeon 5067. Alternatively, when the AE function, the AF function, and the AWB function are provided in the endoscope 5001, the control unit 5063 appropriately calculates an optimum exposure value, focal length, and white balance according to a result of detection processing by the image processing unit 5061, and generates the control signal.

Furthermore, the control unit 5063 causes the display device 5041 to display the surgical site image based on the image signal subjected to the image processing by the image processing unit 5061. At this time, the control unit 5063 recognizes various objects in a surgical site image using various image recognition technologies. For example, the control unit 5063 can recognize the surgical instrument such as forceps, a specific living body site, bleed, mist at the time of using the energy treatment tool 5021, and the like by detecting a shape, color, and the like of an edge of an object included in the surgical site image. When displaying the surgical site image on the display device 5041, the control unit 5063 superimposes and displays various types of surgery support information on the surgical site image using the recognition result. The surgery support information is displayed in a superimposed manner and is presented to the surgeon 5067, so that it is possible to proceed with the surgery more safely and reliably.

The transmission cable 5065 connecting the camera head 5005 and the CCU 5039 is an electric signal cable compatible with electric signal communication, an optical fiber compatible with optical communication, or a composite cable thereof.

Here, in the illustrated example, wired communication is performed using the transmission cable 5065, but in the present disclosure, communication between the camera head 5005 and the CCU 5039 may be performed wirelessly. When the communication between the camera head 5005 and the CCU 5039 is performed wirelessly, it is not necessary to lay the transmission cable 5065 in the operating room. As a result, situation in which the movement of medical staff (e.g., surgeon 5067) in the operating room is hindered by the transmission cable 5065 can be eliminated.

### <1.5 Configuration Example of Endoscope 5001>

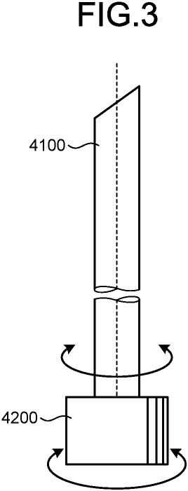

Next, a basic configuration of a forward-oblique viewing endoscope will be described as an example of the endoscope 5001 with reference to FIG. 3. FIG. 3 is a schematic diagram illustrating a configuration of a forward-oblique viewing endoscope 4100 according to the embodiment of the present disclosure.

Specifically, as illustrated in FIG. 3, the forward-oblique viewing endoscope 4100 is attached to the distal end of a camera head 4200. The forward-oblique viewing endoscope 4100 corresponds to the lens barrel 5003 described with reference to FIGS. 1 and 2, and the camera head 4200 corresponds to the camera head 5005 described with reference to FIGS. 1 and 2. The forward-oblique viewing endoscope 4100 and the camera head 4200 are independently rotatable from each other. An actuator is provided between the forward-oblique viewing endoscope 4100 and the camera head 4200 similarly to the joints 5033a, 5033b, and 5033c, and the forward-oblique viewing endoscope 4100 rotates with respect to the camera head 4200 by driving the actuator.

The forward-oblique viewing endoscope 4100 is supported by the support arm device 5027. The support arm device 5027 has a function of holding the forward-oblique viewing endoscope 4100 instead of the scopist and moving the forward-oblique viewing endoscope 4100 such that a desired site can be observed according to the manipulation by the surgeon 5067 or the assistant.

Note that, in the embodiment of the present disclosure, the endoscope 5001 is not limited to the forward-oblique viewing endoscope 4100. For example, the endoscope 5001 may be the forward-viewing endoscope (not illustrated) that captures the front of the distal end of the endoscope, and may further have the function of cutting out the image from a wide-angle image captured by the endoscope (wide-angle/cutout function). Furthermore, for example, the endoscope 5001 may be an endoscope with a distal end bending function (not illustrated) capable of changing the visual field by freely bending the distal end of the endoscope according to the manipulation by the surgeon 5067. Furthermore, for example, the endoscope 5001 may be an endoscope with a simultaneous imaging function in another direction (not illustrated) in which a plurality of camera units having different visual fields is built in the distal end of the endoscope to obtain different images by the cameras.

An example of the endoscopic surgery system 5000 to which the technology according to the present disclosure can be applied has been described above. Note that, here, the endoscopic surgery system 5000 has been described as an example. A system to which the technology according to the present disclosure can be applied is not limited to the example. For example, the technology according to the present disclosure may be applied to a microscopic surgery system.

### <<2. Configuration Example of Medical Observation System 10>>

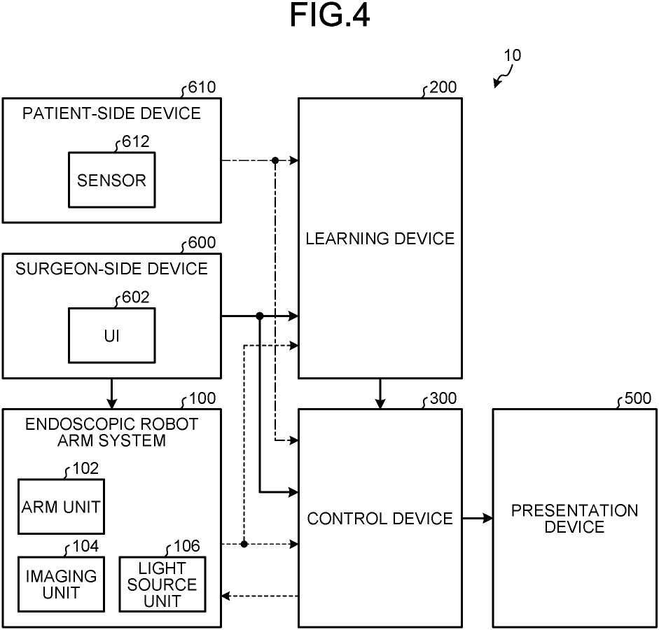

Next, a configuration example of the medical observation system 10 according to the embodiment of the present disclosure that can be combined with the above-described endoscopic surgery system 5000 will be described with reference to FIG. 4. FIG. 4 is a diagram illustrating the configuration example of the medical observation system 10 according to the embodiment of the present disclosure. As illustrated in FIG. 4, the medical observation system 10 mainly includes an endoscopic robot arm system 100, a learning device 200, a control device 300, a presentation device 500, a surgeon-side device 600, and a patient-side device 610. Hereinafter, each device included in the medical observation system 10 will be described.

First, before describing the details of the configuration of the medical observation system 10, an outline of the operation of the medical observation system 10 will be described. In the medical observation system 10, by controlling an arm unit 102 (corresponding to the support arm device 5027 described above) using the endoscopic robot arm system 100, a position of an imaging unit 104 (corresponding to the endoscope 5001 described above) supported by the arm unit 102 can be fixed at a suitable position without manual control. Therefore, according to the medical observation system 10, since the surgical site image can be stably obtained, the surgeon 5067 can smoothly perform the surgery. Note that, in the following description, a person who moves or fixes the position of the endoscope is referred to as the scopist, and the movement of the endoscope 5001 (including transfer, stop, and change in attitude) is referred to as a scope work regardless of manual or mechanical control.

### (Endoscopic Robot Arm System 100)

The endoscopic robot arm system 100 is the arm unit 102 (support arm device 5027) that supports the imaging unit 104 (endoscope 5001), and specifically, as illustrated in FIG. 4, mainly includes the arm unit (medical arm) 102, the imaging unit (medical observation device) 104, and a light source unit 106. Hereinafter, each functional unit included in the endoscopic robot arm system 100 will be described.

The arm unit 102 includes an articulated arm (corresponding to the arm 5031 illustrated in FIG. 1) that has a multilink structure including a plurality of joints and a plurality of links, and can control the position and attitude of the imaging unit 104 (endoscope 5001) provided at the distal end of the arm unit 102 by driving the arm unit 102 within a movable range. Furthermore, the arm unit 102 may have a motion sensor (not illustrated) such as an acceleration sensor, a gyro sensor, and a geomagnetic sensor in order to obtain data of the position and attitude of the arm unit 102.

The imaging unit 104 is provided, for example, at the distal end of the arm unit 102 and captures images of various imaging targets. In this case, the arm unit 102 supports the imaging unit 104. Note that, in the present embodiment, a relay lens that guides light from a subject to the image sensor may be provided at the distal end of the arm unit 102, and the light may be guided to the image sensor in the CCU 5039 by the relay lens. Furthermore, as described above, the imaging unit 104 may be, for example, the forward-oblique viewing endoscope 4100, a forward-viewing endoscope with the wide-angle/cutout function (not illustrated), the endoscope with the distal end bending function (not illustrated), the endoscope with the simultaneous imaging function in another direction (not illustrated), or the microscope, and is not particularly limited.

Furthermore, the imaging unit 104 can capture, for example, an operative field image including various medical instruments (surgical instruments) and organs in the abdominal cavity of the patient. Specifically, the imaging unit 104 is a camera capable of capturing an imaging target in the form of a moving image or a still image, and is preferably a wide-angle camera including a wide-angle optical system. For example, while the angle of view of a normal endoscope is about 80°, the angle of view of the imaging unit 104 according to the present embodiment may be 140°. Note that the angle of view of the imaging unit 104 may be smaller than 140° or may be 140° or more as long as the angle of view exceeds 80°. Furthermore, the imaging unit 104 can transmit an electric signal (image signal) corresponding to the captured image to the control device 300 or the like. Note that, in FIG. 4, the imaging unit 104 is not necessarily included in the endoscopic robot arm system 100, and a mode thereof is not limited as long as the imaging unit is supported by the arm unit 102. Furthermore, the arm unit 102 may support the medical instrument such as the forceps 5023.

Furthermore, in the embodiment of the present disclosure, the imaging unit 104 may be a stereoscopic endoscope capable of performing distance measurement. Alternatively, in the embodiment of the present disclosure, a depth sensor of a time of flight (ToF) system that performs distance measurement using reflection of pulsed light or of a structured light system that performs distance measurement by emitting lattice-shaped pattern light may be provided separately from the imaging unit 104.

Furthermore, in the light source unit 106, the imaging unit 104 irradiates the imaging target with light. The light source unit 106 can be realized by, for example, a light emitting diode (LED) for wide angle lens. For example, the light source unit 106 may be configured by combining a normal LED and a lens so as to diffuse light. Furthermore, the light source unit 106 may have a configuration in which light transmitted through an optical fiber (light guide) is diffused (widened) by the lens. In addition, the light source unit 106 may expand an irradiation range by irradiating the optical fiber itself with light in a plurality of directions. Note that, in FIG. 4, the light source unit 106 is not necessarily included in the endoscopic robot arm system 100, and a mode thereof is not limited as long as the irradiation light can be applied to the subject.

### (Learning Device 200)

The learning device 200 is a device that generates a learning model used when generating autonomous movement control information for causing the endoscopic robot arm system 100 to autonomously move, for example, by a central processing unit (CPU) or a micro processing unit (MPU). Furthermore, the learning model used in the embodiment of the present disclosure is generated by learning a learned model that performs classification of input information and processing according to a classification result based on features of various types of input information. The learning model may be realized by a deep neural network (DNN) or the like that is a multilayer neural network having a plurality of nodes including an input layer, a plurality of intermediate layers (hidden layers), and an output layer. For example, in the generation of the learning model, first, various types of input information are input via the input layer, and extraction of a feature included in the input information is performed in a plurality of intermediate layers connected in series. Next, the learning model can be generated by outputting, via the output layer, various processing results such as the classification result based on the information output by the intermediate layer as output information corresponding to the input information. However, the embodiment of the present disclosure is not limited thereto.

Note that a detailed configuration of the learning device 200 will be described later. Furthermore, the learning device 200 may be a device integrated with at least one of the endoscopic robot arm system 100, the control device 300, the presentation device 500, the surgeon-side device 600, and the patient-side device 610 illustrated in FIG. 4 described above, or may be a separate device. Alternatively, the learning device 200 may be a device provided on a cloud and communicably connected to the endoscopic robot arm system 100, the control device 300, the presentation device 500, the surgeon-side device 600, and the patient-side device 610.

### (Control Device 300)

The control device 300 controls driving of the endoscopic robot arm system 100 based on the learning model generated by the learning device 200 described above. The control device 300 is implemented by, for example, the CPU, the MPU, or the like executing a program (e.g., a program according to the embodiment of the present disclosure) stored in a storage unit to be described later using a random access memory (RAM) or the like as a work area. Furthermore, the control device 300 is a controller, and may be realized by, for example, an integrated circuit such as an application specific integrated circuit (ASIC) or a field programmable gate array (FPGA).

Note that a detailed configuration of the control device 300 will be described later. Furthermore, the control device 300 may be a device integrated with at least one of the endoscopic robot arm system 100, the learning device 200, the presentation device 500, the surgeon-side device 600, and the patient-side device 610 illustrated in FIG. 4 described above, or may be a separate device. Alternatively, the control device 300 may be a device provided on a cloud and communicably connected to the endoscopic robot arm system 100, the learning device 200, the presentation device 500, the surgeon-side device 600, and the patient-side device 610.

### (Presentation Device 500)

Presentation device 500 displays various images. Presentation device 500 displays, for example, an image captured by the imaging unit 104. The presentation device 500 can be, for example, a display including a liquid crystal display (LCD) or an organic electro-luminescence (EL) display. Note that the presentation device 500 may be a device integrated with at least one of the endoscopic robot arm system 100, the learning device 200, the control device 300, the surgeon-side device 600, and the patient-side device 610 illustrated in FIG. 4 described above. Alternatively, the presentation device 500 may be a separate device communicably connected to at least one of the endoscopic robot arm system 100, the learning device 200, the control device 300, the surgeon-side device 600, and the patient-side device 610 in a wired or wireless manner.

### (Surgeon-Side Device 600)

The surgeon-side device 600 is a device installed in a vicinity of the surgeon 5067, and more particularly, for example, a user interface (UI) 602. Specifically, the UI 602 is an input device that receives an input by the surgeon. More specifically, the UI 602 can be a control stick (not illustrated) that receives text input by the surgeon 5067, a button (not illustrated), a keyboard (not illustrated), a foot switch (not illustrated), a touch panel (not illustrated), a master console (not illustrated), or a sound collection device (not illustrated) that receives voice input by the surgeon 5067. In addition, the UI 602 may include a line-of-sight sensor (not illustrated) that detects the line of sight of the surgeon 5067, a motion sensor (not illustrated) that detects an action of the surgeon 5067, and the like, and may receive an input by the movement of the line of sight or action of the surgeon 5067.

### (Patient-Side Device 610)

The patient-side device 610 may be, for example, a device worn (wearable device) on a patient body (not illustrated), and more particularly, for example, a sensor 612. Specifically, the sensor 612 is a sensor that detects biological information of the patient, and can be, for example, various sensors that are directly attached to parts of the patient body to measure the patient’s heart rate, pulse, blood pressure, blood oxygen concentration, brain waves, respiration, perspiration, myoelectric potential, skin temperature, and skin electrical resistance. Furthermore, the sensor 612 may include an imaging device (not illustrated), and in this case, the imaging device may acquire sensing data including information such as the patient’s pulse, muscle movement (expression), eye movement, pupil diameter, and line of sight. Furthermore, the sensor 612 may include a motion sensor (not illustrated) and acquire sensing data including information, for example, patient’s head movement or attitude, and shaking of body.

### 3. Background to Creation of Embodiment of Present Disclosure

In the medical observation system 10 as described above, development for autonomously moving the endoscopic robot arm system 100 is in progress. More specifically, the autonomous movement of the endoscopic robot arm system 100 in the medical observation system 10 can be divided into various levels. These levels include a level at which the surgeon 5067 is guided by the system and a level at which some movements (tasks) in the surgery, such as moving the position of the imaging unit 104 and suturing the surgical site, are autonomously executed by the system. Furthermore, the levels include a level at which movement options in the surgery are automatically generated by the system, and the endoscopic robot arm system 100 performs a movement selected by the doctor from the automatically generated movement options. In the future, it is also conceivable that the endoscopic robot arm system 100 executes all the tasks in the surgery under the monitoring of the doctor or without the monitoring of the doctor.

In the embodiment of the present disclosure described below, it is assumed that the endoscopic robot arm system 100 autonomously executes a task of moving the imaging position of the imaging unit 104 (scope work) instead of the scopist, and the surgeon 5067 performs surgery directly or by remote control with reference to an image captured by the imaging unit 104 after being moved. For example, in the endoscopic surgery, an inappropriate scope work leads to an increase in burden on the surgeon 5067, such as fatigue and cybersickness of the surgeon 5067. Furthermore, since the scope work is difficult and experts are in short supply, the endoscopic robot arm system 100 is required to autonomously perform the scope work appropriately. Therefore, it is required to obtain a model of an appropriate scope work (movement) for the autonomous movement of the endoscopic robot arm system 100.

However, since preference and expected degree of scope work differ depending on the surgeon 5067 and the like, it is difficult to achieve a correct answer to the scope work. In other words, since the quality of the scope work is related to a human sensitivity (surgeon 5067, scopist, etc.), it is difficult to quantitatively evaluate the quality of the scope work and model an appropriate scope work. Therefore, it is conceivable to generate a learning model of the appropriate scope work by inputting a large amount of data regarding surgical operations and the like and corresponding surgical actions by the surgeon 5067 and scope works by the scopist to the learning device and causing the learning device to perform machine learning.

However, since the body shape, organ form, organ position, and the like are different for each patient, it is practically difficult to acquire movement data of the scope work covering a wider range of situations in a clinical field (movement data including information indicating movement of the arm unit 102, organ form of the patient, organ position, etc.). In addition, in a medical field, there are restrictions on devices and time that can be used, and further, it is necessary to protect patient privacy. Thus, it is difficult to acquire a large amount of movement data of the scope work.

Therefore, in view of the above circumstances, the inventor has conceived using reinforcement learning, which is one method of machine learning, to acquire a learning model covering a wider range of situations. Now, each method of machine learning will be described.

There are a plurality of different methods in machine learning such as supervised learning, unsupervised learning, and reinforcement learning.

Specifically, in the supervised learning, a plurality of combinations of input data and desirable output data (correct answer data) (training data) to the input data are prepared in advance, and the learning device (determination unit) performs machine learning of these pieces of data so as to derive a relationship between the input data and the training data that can reproduce the combinations. For example, the supervised learning is used to acquire a learning model for predicting a next movement (desirable output data) using the movement and state of the arm unit 102 in a predetermined period as the input data.

Next, in a model generated by unsupervised learning, it is possible to extract similar feature amounts between the input data without defining the desirable output data (correct answer data). The unsupervised learning is used for clustering similar data from a data group or extracting a data structure.

Furthermore, the reinforcement learning is similar to the above-described supervised learning in that the reinforcement learning is used for acquiring the desirable output data (correct answer data) with respect to the input data. However, in the reinforcement learning, instead of learning using the combination of the input data and the desirable output data (training data) to the input data as in the supervised learning, learning by trial and error is performed using three elements (state, action, reward). Specifically, in the reinforcement learning, when an agent (e.g., arm unit 102) performs a certain “action” in a certain “state”, a process of giving a “reward” is repeated when the action is the correct answer. Then, in the reinforcement learning, by repeating trial and error so as to increase the reward to be given, it is possible to acquire a learning model capable of determining an appropriate “action” in various “states”.

The reinforcement learning will be described with a more specific example. Here, as an example, it is considered to acquire a learning model that enables a wheeled platform robot on which an inverted pendulum having one degree of freedom of rotation is mounted to perform a movement in which the inverted pendulum maintains an inverted state. The wheeled platform robot is provided with a sensor capable of acquiring a speed, acceleration, and an angle of the inverted pendulum of the wheeled platform robot itself in real time. In this case, three pieces of sensing data of the speed, the acceleration, and the angle of the inverted pendulum of the wheeled platform robot are input as the “state” to the learning device that performs the reinforcement learning. Then, the learning device outputs next acceleration of the wheeled platform robot as “action” based on the “state” input. At this time, the action is determined by probability-based selection. In other words, even when the same “state” is input, the same “action” may not be selected every time by the learning device, and thus trial and error occur. Further, the “action” selected by the learning device is executed by the wheeled platform robot, and the “state” further changes.

Furthermore, in this example, the “reward” given to the “state” is designed such that the “reward” (value) increases when the desired “state”, i.e., the inverted state of the inverted pendulum is achieved. For example, the “reward” is designed such that a reward value is 100 when the inverted pendulum is in the inverted state, and the reward value is decreased by 20 every time the inverted pendulum is shifted from the inverted state by one degree. Therefore, since an “(immediate) reward” to be given to the “state” caused by various “actions” selectable in the current “state” is known, the learning device selects the next “action” that can expect to maximize a total reward in the future from the “actions” selectable in the current “state”. Then, by repeating the trial and error, the learning device performs learning to reinforce easy selection of the “action” that can maximize the total reward in the future.

As described above, instead of preparing the correct answer data in advance as the training data, a learning model that outputs an “action” that maximizes the total “reward” given in the future according to a result of the “action” is acquired through the reinforcement learning, which is different from the supervised learning.

In the embodiment of the present disclosure created by the present inventor, a learning model covering a wider range of situations can be acquired by using the reinforcement learning as described above. However, in the example of the wheeled platform robot on which the inverted pendulum is mounted, it is easy to define the “reward” since the correct answer to the “state” is clear. On the other hand, as described above, since the preference and expected degree of scope work differ depending on the surgeon 5067 and the like, it is difficult to find the correct answer to the “state”. Accordingly, it is not easy to define the “reward” for appropriate scope work. Therefore, it is not possible to acquire the learning model for appropriate autonomous movement of the scope work only by using the reinforcement learning.

Therefore, the present inventor has uniquely conceived acquisition of a definition of “reward” for the reinforcement learning by machine learning. In the embodiment of the present disclosure created by the inventor of the present invention, first, for example, movement data of a clinical scope work and state data such as a position of the endoscope obtained in each movement are input to the learning device as input data (first input data) and training data (first training data), respectively, and the supervised learning is performed, thereby generating a learning model for appropriate autonomous movement of the scope work. Next, in the present embodiment, movement data of the clinical scope work and corresponding score are input to the learning device as input data (second input data) and training data (second training data), respectively, and the supervised learning is performed, thereby generating a learning model that defines a “reward” given to the scope work. Furthermore, in the present embodiment, the reinforcement learning to reinforce the learning model for appropriate autonomous movement of the scope work is performed using the “reward” obtained by the learning model that defines the “reward” according to, for example, input data that is virtual clinical data (third input data). In other words, according to the present embodiment, by combining the supervised learning and the reinforcement learning, it is possible to efficiently acquire the learning model for autonomously performing the scope work in consideration of human sensitivity while covering a wider range of situations even in a case where only a small amount of clinical data can be obtained. Hereinafter, details of the embodiment of the present disclosure created by the present inventor will be sequentially described.

Note that, in the present specification, the virtual clinical data is data acquired through surgical simulations under various cases and conditions such as positions and shapes of organs, whereas the clinical data is acquired when the doctor actually performs surgery on the surgical site of the patient.

### 4. Embodiment

### <4.1 Detailed Configuration of Learning Device 200>

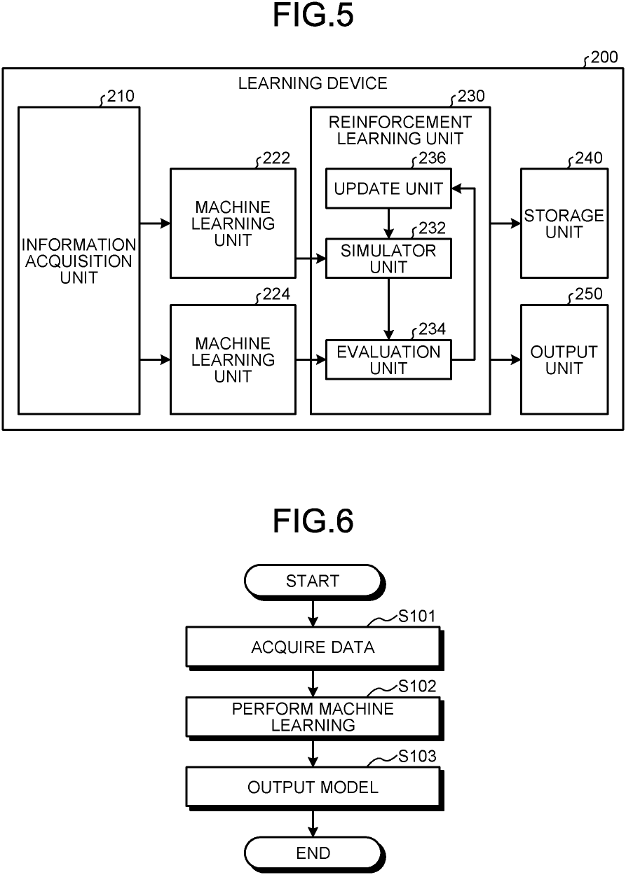

First, a detailed configuration example of the learning device 200 according to the embodiment of the present disclosure will be described with reference to FIG. 5. FIG. 5 is a block diagram illustrating an example of a configuration of the learning device 200 according to the present embodiment. The learning device 200 can generate and reinforce a learning model used for controlling autonomous movement of the endoscopic robot arm system 100. Specifically, as illustrated in FIG. 5, the learning device 200 mainly includes an information acquisition unit 210, a machine learning unit (first determination unit) 222, a machine learning unit (second determination unit) 224, a reinforcement learning unit 230, a storage unit 240, and an output unit 250. Hereinafter, details of each functional unit of the learning device 200 will be sequentially described.

### (Information Acquisition Unit 210)

The information acquisition unit 210 can acquire various types of data regarding a state of the endoscopic robot arm system 100, input information from the surgeon 5067 and the like, a state of the patient (not illustrated), and the like from the endoscopic robot arm system 100, the UI 602, and the sensor 612 described above. Further, the information acquisition unit 210 outputs the data acquired to the machine learning unit 222 and the machine learning unit 224 described later.

In the present embodiment, examples of the data include the image data such as an image acquired by the imaging unit 104. In the present embodiment, the data acquired by the information acquisition unit 212 preferably includes at least the image data. Note that, in the present embodiment, the image data is preferably image data (clinical data) acquired at the time of actual surgery but is not limited thereto. For example, the image data may be image data (simulated clinical data) acquired at the time of simulated surgery using a medical phantom (model), or may be image data (virtual clinical data) acquired by a surgery simulator represented by three-dimensional graphics or the like. Furthermore, in the present embodiment, the image data is not necessarily limited to the image of the medical instrument (not illustrated) or the organ, and for example, may include only the image of the medical instrument or only the image of the organ. Furthermore, in the present embodiment, the image data is not limited to raw data acquired by the imaging unit 104, and may be, for example, data obtained by applying processing (adjustment of luminance and saturation, extraction of information on position, attitude, and type of the medical instrument or organ from the image (surgical site information), semantic segmentation, etc.) to the raw data acquired by the imaging unit 104. In addition, in the present embodiment, information such as recognized or estimated sequence or context of the surgery (e.g., metadata) may be associated with the image data. Furthermore, in the present embodiment, the data may include information on imaging conditions (e.g., focus, imaging area, and imaging direction) corresponding to the image acquired by the imaging unit 104.

Note that, in the present specification, the clinical data means data actually acquired when the doctor performs surgery on the patient’s surgical site. In addition, the simulated clinical data means data acquired when the doctor or the like performs the simulated operation using the medical phantom (model) or the like. In addition, as described above, the virtual clinical data means data acquired when surgery simulation is performed on various cases or under conditions such as positions and shapes of organs.

Furthermore, in the present embodiment, the data may be, for example, information such as the distal end or the joint (not illustrated) of the arm unit 102, and the imaging position and attitude of the imaging unit 104. In the present embodiment, these pieces of data can be acquired based on joint angles and link lengths of a joint 5033 and a link 5035 (a plurality of elements) included in the arm unit 102 of the endoscopic robot arm system 100 at the time of manual movement by the scopist or autonomous movement. Alternatively, in the present embodiment, the data may be acquired from the motion sensor provided in the endoscopic robot arm system 100. Note that, as a manual manipulation of the endoscopic robot arm system 100, a method in which the scopist operates the UI 602 may be used, or a method in which the scopist physically grips a part of the arm unit 102 directly and applies a force, so that the arm unit 102 passively operates according to the force may be used. Furthermore, in the present embodiment, the data may be the type, position, attitude, and the like of the medical instrument (not illustrated) supported by the arm unit 102. Note that, in the present embodiment, the above-described data is preferably data acquired at the time of actual surgery (clinical data), but the data may also include simulated clinical data and virtual clinical data.

Furthermore, in the present embodiment, the data may be information such as the position and attitude of the organ that will be the surgical site, position information of the entire surgical site (e.g., depth information), and more particularly, information indicating positional relationship between the organ that is the surgical site and the medical instrument.

Furthermore, in the present embodiment, the data may be, for example, biological information (patient information) of the patient (not illustrated). More specifically, examples of the biological information are patient’s line of sight, blinking, heartbeat, pulse, blood pressure, amount of oxygen in hair, brain waves, respiration, sweating, myoelectric potential, skin temperature, skin electrical resistance, spoken voice, posture, and motion (e.g., shaking of head or body). These pieces of biological information are preferably general clinical data recorded in the endoscopic surgery.

Furthermore, in the present embodiment, the data may be, for example, a score of the scope work. More specifically, the score may be a subjective evaluation score of the scope work entered via the UI 602 by a medical worker (user), such as the surgeon 5067 or the scopist. For example, an expert such as the doctor can obtain the subjective evaluation score by reviewing the scope work (e.g., image captured by the imaging unit 104) and inputting evaluation of the scope work based on an evaluation scale (e.g., numerical scale for evaluation) used when scoring the scope work and capability of the scopist in the medical field, such as a Nilsson score. In the present embodiment, by using such an evaluation scale, it is possible to acquire evaluation information (subjective evaluation) based on the sensitivity of the medical worker. Note that, in the present embodiment, the evaluation scale is not particularly limited to a conventionally existing evaluation scale such as the Nilsson score, may be a newly and independently determined evaluation scale, and is not particularly limited.

### (Machine Learning Unit 222, Machine Learning Unit 224)

The machine learning unit 222 and the machine learning unit 224 can generate an autonomous movement control model and a reward model for causing the endoscopic robot arm system 100 to autonomously move by performing machine learning using the data output from the above-described information acquisition unit 210. Then, the machine learning unit 222 and the machine learning unit 224 output the generated autonomous movement control model and reward model to the reinforcement learning unit 230 described later. The generated reward model is used when the reinforcement learning unit 230 performs reinforcement learning on the generated autonomous movement control model.

The machine learning unit 222 and the machine learning unit 224 are, for example, learning devices that perform supervised learning such as support vector regression or deep neural network (DNN). Furthermore, in the present embodiment, the machine learning unit 222 and the machine learning unit 224 may use an algorithm of a regression method using a structure such as a Gaussian process regression model, a decision tree, or a fuzzy rule that can be handled more analytically, and thus the algorithm is not particularly limited.

Specifically, the machine learning unit 222 acquires, as the input data (first input data), the distal end and the joint (not illustrated) of the arm unit 102, the imaging position and attitude of the imaging unit (endoscope) 104, the type, position and attitude of the medical instrument supported by the arm unit 102, the position and attitude of the organ, the image acquired by the imaging unit 104 (e.g., endoscopic image), information indicating the positional relationship between the organ and the medical instrument (depth information), biological information (vital sign) of the patient, and the like. Furthermore, the machine learning unit 222 acquires, as the training data, the imaging position and attitude of the imaging unit (endoscope) 104, the imaging area and the imaging direction of the imaging unit 104, and the like. The data input to the machine learning unit 222 is preferably the clinical data acquired in clinical work, but the data may also include the simulated clinical data and the virtual clinical data. Then, the machine learning unit 222 generates the autonomous movement control model for causing the endoscopic robot arm system 100 to autonomously move by performing machine learning on these pieces of input data and training data. The autonomous movement control model can output information regarding movement of the endoscopic robot arm system 100 according to the input data (information regarding the distal end of the arm unit 102, or the position, attitude, speed, angular velocity, acceleration, and each acceleration of the imaging unit 104, imaging conditions of the image (e.g., subject (e.g., medical instrument), imaging area, and imaging direction).

For example, by using the imaging position and attitude of the imaging unit (endoscope) 104 as the input data, the machine learning unit 222 can acquire a learning model for determining a next imaging position and the like of the imaging unit 104 based on the current state of the imaging unit 104. Furthermore, by using the type, position, and attitude of the medical instrument as the input data, the machine learning unit 222 can acquire a learning model for determining the imaging area or the like of the imaging unit 104 according to a treatment (e.g., a surgical procedure). Furthermore, by using the position of the organ as the input data, the machine learning unit 222 can acquire a learning model for determining the imaging area or the like of the imaging unit 104 according to the organ. In addition, by using the information indicating the positional relationship between the organ and the medical instrument as the input data, the machine learning unit 222 can acquire a learning model for predicting a next treatment based on a difference in the positional relationship and determining an appropriate imaging distance or the like. Furthermore, by using the biological information of the patient as the input data, the machine learning unit 222 can acquire a learning model for determining treatment according to the state of the patient.

The machine learning unit 224 acquires the image data captured (e.g., endoscopic image) by the imaging unit 104, the biological information (vital signs) of the patient, and the like as the input data (second input data). Furthermore, the machine learning unit 224 acquires a subjective evaluation result (score) (evaluation score) of the scope work as the training data. Then, the machine learning unit 224 generates a reward model that gives a score to the movement of the endoscopic robot arm system 100 (scope work) by performing machine learning on these pieces of input data and training data. In the present embodiment, since the input data input to the machine learning unit 224 is the clinical data generally acquired for the recording purpose in the endoscopic surgery, it is easy to collect the input data, and there is no burden on the medical site. Furthermore, in the present embodiment, since the evaluation score, which is the training data input to the machine learning unit 224, is also the clinical data that is generally recorded in the endoscopic surgery in order to evaluate the scopist, and data collection can be facilitated by using an indicator familiar to the doctor or the like who evaluates, it is possible to suppress an increase in a burden on the medical site. Therefore, in the machine learning for generating the reward model according to the present embodiment, it is possible to realize learning using a large amount of data because data collection is easy.

### (Reinforcement Learning Unit 230)

The reinforcement learning unit 230 performs the reinforcement learning on the autonomous movement control model in the reinforcement learning unit 230 using the reward model. As described above, the reinforcement learning is a learning method using three elements that are the state, movement (action), and reward, and is a method for learning an optimal movement in various states by repeating the process of giving a reward to a certain movement in a certain state when the movement is correct.

Specifically, as illustrated in FIG. 5, the reinforcement learning unit 230 includes a simulator unit 232, an evaluation unit 234, and an update unit 236. Specifically, the simulator unit 232 uses the autonomous movement control model output from the machine learning unit 222 to determine information (acquire virtual clinical data) regarding movement of the endoscopic robot arm system 100 (autonomous movement) under various simulation conditions (e.g., surgical site information in various cases and patient biological information) (e.g., distal end of the arm unit 102 or the position, attitude, speed, angular velocity, acceleration, and each acceleration of the imaging unit 104, imaging conditions of the image (e.g., subject (e.g., medical instrument), imaging area, and imaging direction). Then, the simulator unit 232 outputs information related to the determination to the evaluation unit 234. Next, the evaluation unit 234 uses the reward model output from the machine learning unit 224 to determine a reward for the movement (third input data) of the endoscopic robot arm system 100 under a certain condition (state). Furthermore, the update unit 236 determines (updates) a next movement of the endoscopic robot arm system 100 so as to maximize the total reward in the future, and outputs the movement determined to the simulator unit 232. Furthermore, the simulator unit 232 outputs, to the evaluation unit 234, state information (e.g., distal end of the arm unit 102, or position, attitude, image data, and the like of the imaging unit 104) that is a result of the updated movement. Furthermore, the evaluation unit 234 determines a reward for the movement of the endoscopic robot arm system 100 based on the state output.

In other words, in the present embodiment, the reinforcement learning unit 230 determines the movement of the endoscopic robot arm system 100 using the autonomous movement control model acquired from the machine learning unit 222 as the initial state, but thereafter, updates the movement of the endoscopic robot arm system 100 using the reward model.

In the present embodiment, reinforcement learning to reinforce the learning model for the appropriate autonomous movement of the scope work can be performed using the “reward” obtained by the reward model that defines the “reward” obtained by the machine learning unit 224. In other words, according to the present embodiment, by combining the supervised learning and the reinforcement learning, it is possible to efficiently acquire the learning model for autonomously performing the scope work in consideration of human sensitivity while covering a wider range of situations even in a case where only a small amount of clinical data can be obtained.

Note that, in the present embodiment, the reinforcement learning unit 230 is not limited to the deep neural network (DNN), for example, and other known reinforcement learning methods (Q-Learning, Sarsa, Monte Carlo, Actor-Critic) may be used.

### (Storage Unit 240)

The storage unit 240 can store various types of information. The storage unit 240 is realized by, for example, a semiconductor memory element such as a random access memory (RAM) or a flash memory, or a storage device such as a hard disk or an optical disk.

### (Output Unit 250)

The output unit 250 can output the learning model (autonomous movement control model) output from the reinforcement learning unit 230 to the control device 300 described later.

Note that, in the present embodiment, the detailed configuration of the learning device 200 is not limited to the configuration illustrated in FIG. 5. In the present embodiment, the learning device 200 may include, for example, a recognition unit (not illustrated) that recognizes the type, position, attitude, and the like of the medical instrument (not illustrated) used by the surgeon 5067 by using, for example, image analysis or the like from a plurality of pieces of data output from the information acquisition unit 212. Furthermore, the learning device 200 may include, for example, the recognition unit (not illustrated) that recognizes the type, position, attitude, and the like of the organ of the surgical site to be treated by the surgeon 5067 by using, for example, image analysis and the like from the plurality of pieces of data output from the information acquisition unit 212.

### <4.2 Method for Generating Autonomous Movement Control Model>

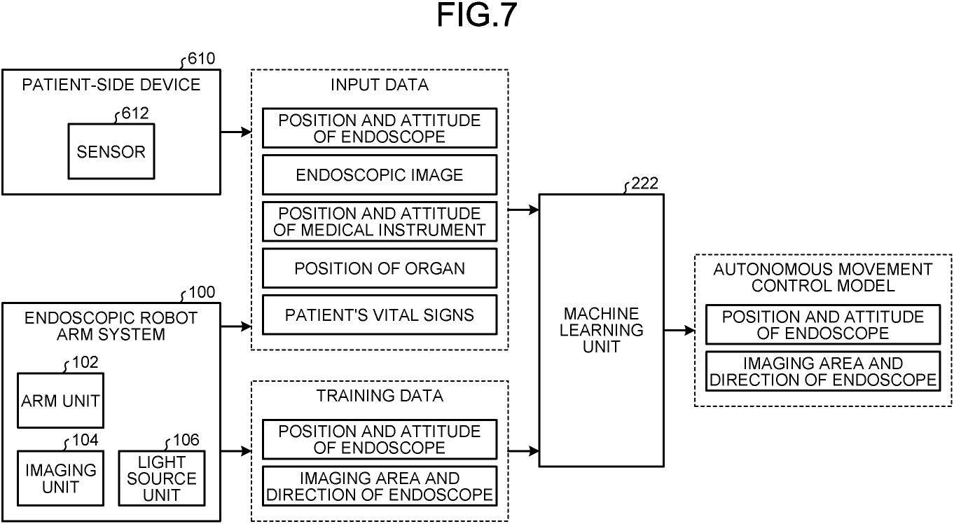

Next, a method for generating the autonomous movement control model according to the present embodiment will be described with reference to FIGS. 6 and 7. FIG. 6 is a flowchart illustrating an example of the method for generating the model according to the present embodiment, and FIG. 7 is an explanatory diagram illustrating an example of the method for generating the autonomous movement control model according to the present embodiment. Specifically, as illustrated in FIG. 6, the method for generating the autonomous movement control model according to the present embodiment includes a plurality of steps from Step S101 to Step S103. Details of each of these steps will be described below.

First, as illustrated in FIG. 7, the learning device 200 acquires various types of data regarding the state of the endoscopic robot arm system 100, the state of the patient (not illustrated), and the like from the endoscopic robot arm system 100 and the sensor 612 (Step S101).

Then, as illustrated in FIG. 7, for example, the imaging position and attitude of the imaging unit (endoscope) 104, the position and attitude of the medical instrument, the position and attitude of the organ, the image data (e.g., endoscopic image) acquired by the imaging unit 104, the biological information (vital signs) of the patient, and the like among the data acquired in Step S101 are used as the input data (first input data), and the learning device 200 performs machine learning using the imaging position, attitude, imaging area, imaging direction, and the like of the imaging unit (endoscope) 104 as the training data (Step S102). Specifically, the learning device 200 acquires, for example, information of a three-dimensional position (x, y, z) of the imaging unit (endoscope) 104 in a plurality of pieces of the clinical data and a variance (σₓ², σ_{y}², σ_{z}²) indicating certainty, and performs machine learning using the input data associated with the training data that is the information of the three-dimensional position where the above variance is faint (nearly 0) as the training data.

Then, the learning device 200 outputs the autonomous movement control model (Step S103). The autonomous movement control model can output, for example, information regarding the imaging position, attitude, imaging area, imaging direction, and the like of the imaging unit (endoscope) 104. Specifically, the autonomous movement control model can output, for example, the information of the three-dimensional position (x, y, z) of the imaging unit (endoscope) 104 and the variance (σₓ², σ_{y}², σ_{z}²) indicating its certainty.

Note that, in the present embodiment, the attitude of the imaging unit (endoscope) 104 is restrained inside body around the affected part when the imaging unit 104 is the forward-viewing endoscope, and thus does not need to be considered. When the imaging unit 104 is an endoscope with the distal end bending function (not illustrated) capable of changing the field of view by freely bending one distal end, it is preferable to add information regarding the attitude of the imaging unit (endoscope) 104.

Furthermore, in the present embodiment, the input data and the training data at the time of generating the autonomous movement control model are not limited to the above-described data, and a plurality of pieces of data may be used in combination in each of the input data and the training data. In addition, the data output from the autonomous movement control model is not limited to the above-described data.

### <4.3 Method for Generating Reward Model>

Next, a method for generating the reward model according to the present embodiment will be described. Note that the learning device 200 that generates the reward model is similar to the learning device 200 according to the present embodiment described with reference to FIG. 5, and thus description thereof is omitted here.

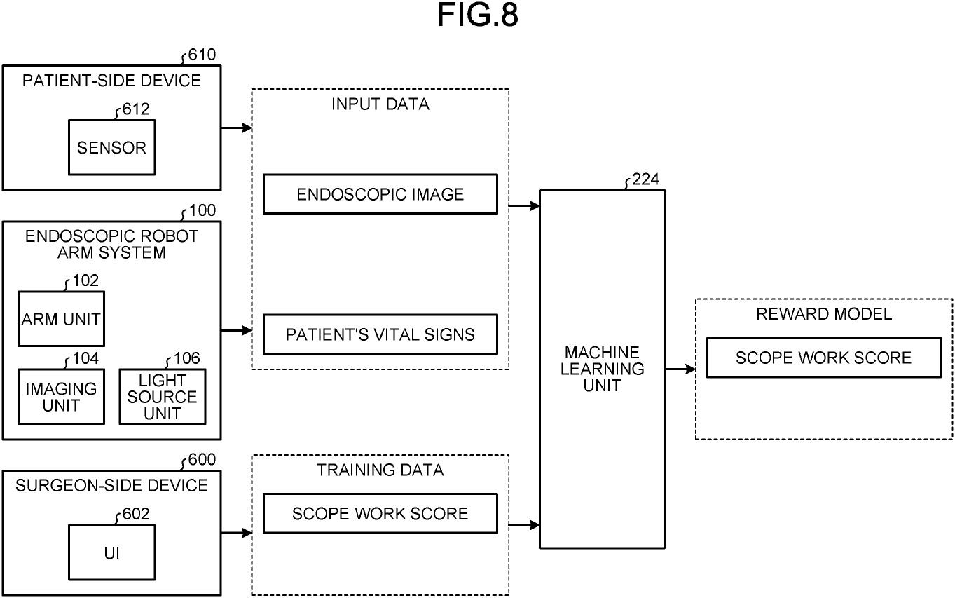

First, the method for generating the reward model according to the present embodiment will be described with reference to FIGS. 6 and 8. FIG. 8 is an explanatory diagram illustrating an example of the method for generating the reward model according to the present embodiment. Specifically, as illustrated in FIG. 6, the method for generating the reward model according to the present embodiment includes a plurality of steps from Step S101 to Step S103, similarly to the generation of the autonomous movement control model. Details of these steps according to the present embodiment will be described below.

First, as illustrated in FIG. 8, the learning device 200 acquires, from the endoscopic robot arm system 100, the UI 602, and the sensor 612, several hundreds of pieces of the input data (second input data), for example, such as the image data (e.g., endoscopic image) acquired by the imaging unit 104 and the evaluation score of the scope work (Step S101). Note that, in the present embodiment, it is preferable to acquire at least a set of image data acquired by the imaging unit 104 and the evaluation score such as the Nilsson score associated with the image data. Since the image data is relatively easily acquired in the endoscopic surgery and is also acquired at the time of generating the autonomous movement control model, it is preferable to generate the reward model using the image data in the present embodiment. In addition, since the Nilsson score or the like is a relatively frequently used indicator, the burden on the doctor can be reduced.

Then, as illustrated in FIG. 8, the learning device 200 performs machine learning using the image data (e.g., endoscopic image) acquired by the imaging unit 104, the biological information (vital signs) of the patient, and the like among the data acquired in Step S101 described above as the input data (second input data) and using the evaluation score of the scope work as the training data (Step S102).

Then, the learning device 200 outputs the reward model (Step S103). Specifically, as illustrated in FIG. 8, the reward model can output the evaluation score of the scope work.

Note that, in the present embodiment, the input data at the time of generating the reward model is not limited to the above-described data, and a plurality of pieces of data may be used in combination.

As described above, in the present embodiment, the “reward” model reflecting human sensitivity can be generated by supervised learning using the clinical data that is relatively easily acquired. Therefore, according to the present embodiment, as described below, it is possible to perform reinforcement learning using the reward model, and thus, it is possible to acquire a learning model that enables the autonomous movement reflecting human sensitivity.

### <4.4 Method for Reinforcing Autonomous Movement Control Model>

Next, a method for reinforcing the autonomous movement control model according to the present embodiment will be described. Note that the learning device 200 that reinforces the autonomous movement control model is similar to the learning device 200 according to the present embodiment described with reference to FIG. 5, and thus the description thereof will be omitted here.

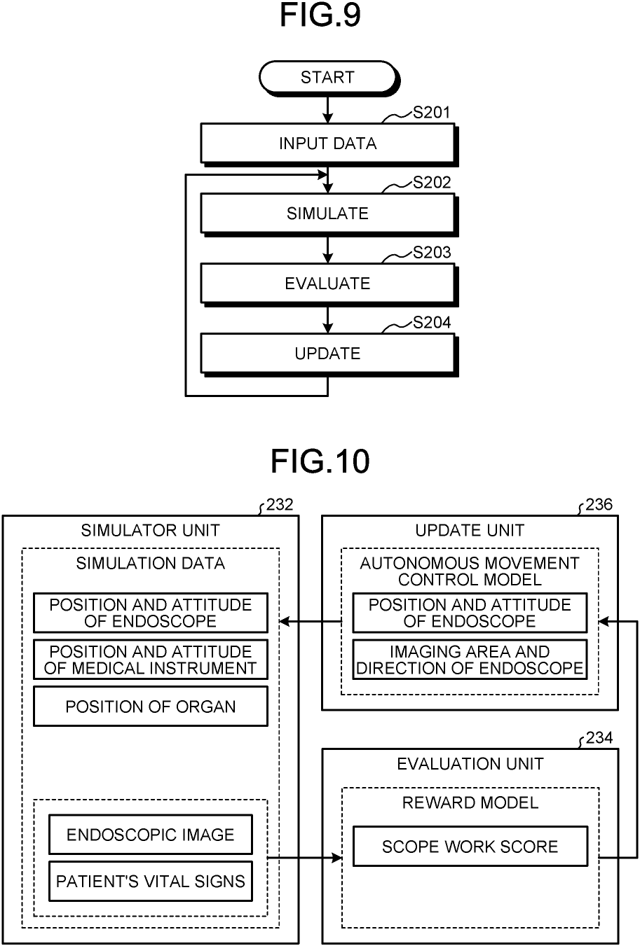

First, the method for reinforcing the autonomous movement control model according to the present embodiment will be described with reference to FIGS. 9 and 10. FIG. 9 is a flowchart illustrating an example of the reinforcement learning according to the present embodiment, and FIG. 10 is an explanatory diagram illustrating an example of reinforcement learning according to the present embodiment. Specifically, as illustrated in FIG. 9, the method for reinforcing the autonomous movement control model according to the present embodiment includes a plurality of steps from Step S201 to Step S204. Details of each of these steps will be described below.

First, when executing the simulation, the learning device 200 acquires data of various cases as simulation conditions (Step S201). For example, the learning device 200 acquires data in consideration of differences in patient’s body shape, size and hardness of the organ, amount of visceral fat, and the like.

Next, the learning device 200 performs simulation using the autonomous movement control model (Step S202). Specifically, the learning device 200 determines information regarding the movement of the endoscopic robot arm system 100 (autonomous movement) (e.g., imaging position and attitude of the imaging unit (endoscope) 104, position and attitude of the medical instrument (not illustrated), etc.) in the simulation conditions based on the data acquired in Step S201 described above. Then, the learning device 200 acquires, by simulation, information regarding the state that is a result of the movement of the endoscopic robot arm system 100 (e.g., imaging position and attitude of the imaging unit (endoscope) 104, position and attitude of the medical instrument (not illustrated), position of the organ, image data by the imaging unit (endoscope), and patient’s vital signs).

Next, the learning device 200 determines evaluation (reward) for the movement (virtual clinical data) of the endoscopic robot arm system 100 using the reward model (Step S203) .

Then, the learning device 200 determines (updates) the next movement of the endoscopic robot arm system 100 so as to maximize the total reward in the future (Step S204).

The learning device 200 can perform reinforcement learning by the neural network using, for example, a policy gradient method. Specifically, in the present embodiment, the movement of the endoscopic robot arm system 100 at a certain time point can be defined by using the policy gradient method. More particularly, when a policy function π(a|s) indicating a probability of the movement of the endoscopic robot arm system 100 is used, whereas a state s is an input and an action probability a is the next selectable action (probability for each of three degrees of freedom is indicated in the case of the three degree of freedom). Therefore, since the policy function itself has a neural network structure, when a parameter θ (weight or bias) of the neural network is used, the parameter θ can be updated by the following Expression (1) using the policy gradient method.

$$\begin{matrix}{θ}_{t + 1} ← {θ}_{t} + α {∇}_{θ} J \left(θ\right) \\ w h e r e J \left(θ\right) = \sum π \left(a \left|s\right)\right) {Q}^{{π}_{θ}} \left(s , a\right)\end{matrix} \qquad \text{(1)}$$

Note that α indicates a learning rate, and J (θ) is an objective function to be optimized and corresponds to an expected value of a cumulative reward (total reward). Q^{πθ}(s,a) indicates a value of the action a that can be selected in the state s. Note that the policy function π(a|s) can be treated as a normal distribution function expressed by an average and a variance.

In an update using Expression (1), a differential value ∇_{θ}J (θ) is required, but approximation is possible by the following Expression (2) using the policy gradient theorem. Here, rₜ is a score obtained by the above-described reward model.

$${∇}_{\text{θ}} J \left(θ\right) \approx {\sum }_{t = t}^{T} {∇}_{θ} log {π}_{θ} \left({a}_{t} \left|{s}_{t}\right)\right) {r}_{t} \qquad \text{(2)}$$

As described above, in the present embodiment, by using the reinforcement learning, it is possible to acquire a learning model covering a wider range of situations even when there is a small amount data available through the clinical work. Furthermore, in the present embodiment, since it is possible to perform the reinforcement learning using the reward model obtained by the supervised learning using the evaluation score, it is possible to obtain the learning model that enables autonomous movement reflecting human sensitivity. In other words, according to the present embodiment, by combining the supervised learning and the reinforcement learning, it is possible to efficiently acquire the learning model for autonomously performing the scope work in consideration of human sensitivity while covering a wider range of situations even in a case where only a small amount of clinical data can be obtained.

### <4.5 Detailed Configuration of Control Device 300>

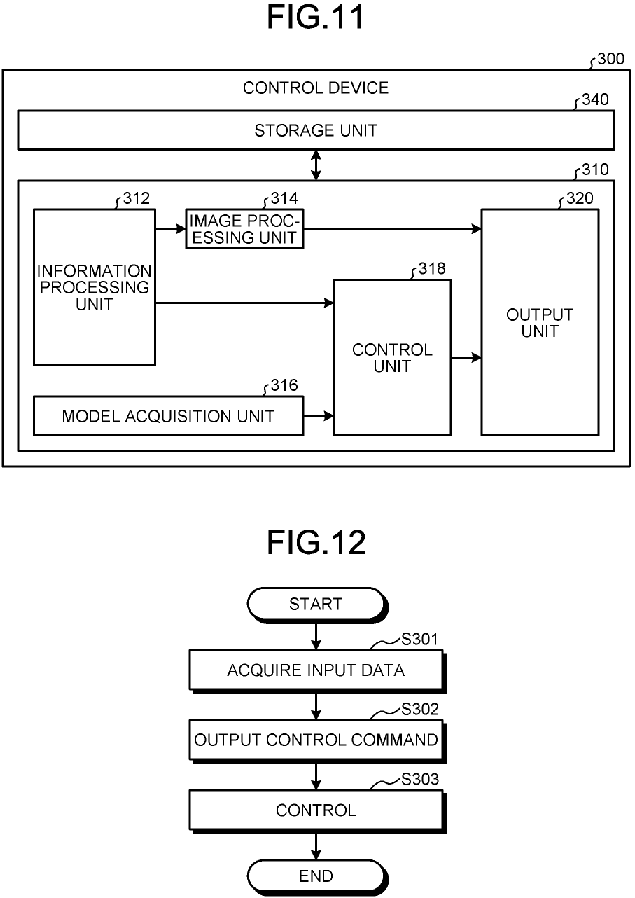

Next, a detailed configuration example of the control device 300 according to the embodiment of the present disclosure will be described with reference to FIG. 11. FIG. 11 is a block diagram illustrating an example of a configuration of the control device 300 according to the present embodiment. The control device 300 can autonomously control the endoscopic robot arm system 100 using the reinforced autonomous movement control model. Specifically, as illustrated in FIG. 11, the control device 300 mainly includes a processing unit 310 and a storage unit 340. Hereinafter, details of each functional unit of the control device 300 will be sequentially described.

### (Processing Unit 310)

As illustrated in FIG. 11, the processing unit 310 mainly includes an information acquisition unit 312, an image processing unit 314, a model acquisition unit 316, a control unit 318, and an output unit 320.

The information acquisition unit 312 can acquire various types of data regarding the state of the endoscopic robot arm system 100 (positions and attitudes of the arm unit 102 and imaging unit 104, position and attitude of imaging unit 104, etc.), the position and attitude of the medical instrument (not illustrated), the position of the organ, the position information on the entire surgical site (depth information), the state of the patient (not illustrated) (vital signs), and the like in real time from the endoscopic robot arm system 100, the UI 202, and the sensor 612 described above. Furthermore, the information acquisition unit 312 outputs the acquired data to the image processing unit 314 and the control unit 318 described later.

The image processing unit 314 can execute various processes on the image captured by the imaging unit 104. Specifically, for example, the image processing unit 314 may generate a new image by cutting out and enlarging a display target area in the image captured by the imaging unit 104. Then, the generated image is output to the presentation device 500 via the output unit 320 described later.

The model acquisition unit 316 can acquire and store the reinforced autonomous movement control model from the learning device 200, and output the reinforced autonomous movement control model to the control unit 318 described later.

Based on the data from the information acquisition unit 312, the control unit 318 generates a control command u to be given to the endoscopic robot arm system 100, using the acquired reinforced autonomous movement control model, for controlling the driving of the arm unit 102, the imaging unit 104 (e.g., the control unit 318 controls an amount of current supplied to the motor in the actuator of the joint to control a rotation speed of the motor and control a rotation angle and the generated torque of the joint ), and the imaging conditions of the imaging unit 104 (e.g., imaging area, direction, focus, magnification ratio, etc.). The determined control command is output to the endoscopic robot arm system 100 via the output unit 320 described later.

At this time, for example, when a value such as a variance value is obtained by the reinforced autonomous movement control model, the control unit 318 may adjust a target value obtained by the autonomous movement control model according to the variance value or the like (e.g., reduction of a movement speed for safety.).

The output unit 326 can output an image processed by the image processing unit 314 to the presentation device 500, and can output the control command output from the control unit 318 to the endoscopic robot arm system 100.

### (Storage Unit 340)

The storage unit 340 can store various types of information. The storage unit 340 is realized by, for example, a semiconductor memory element such as a RAM or a flash memory, or a storage device such as a hard disk or an optical disk.

In the present embodiment, a detailed configuration of the control device 300 is not limited to the configuration illustrated in FIG. 11. In the present embodiment, the control device 300 may include, for example, a recognition unit (not illustrated) that recognizes the type, position, attitude, and the like of the medical instrument (not illustrated) used by the surgeon 5067 by using, for example, image analysis or the like from a plurality of pieces of data output from the information acquisition unit 312. Furthermore, the control device 300 may include, for example, a recognition unit (not illustrated) that recognizes the type, position, attitude, and the like of the organ of the surgical site to be treated by the surgeon 5067 by using, for example, image analysis or the like from the plurality of pieces of data output from the information acquisition unit 312. Note that, as described above, in the present embodiment, the control device 300 may be a device integrated with the above-described endoscopic robot arm system 100 illustrated in FIG. 4 or the like, or may be a separate device, and is not particularly limited.

### <4.6 Control Method>

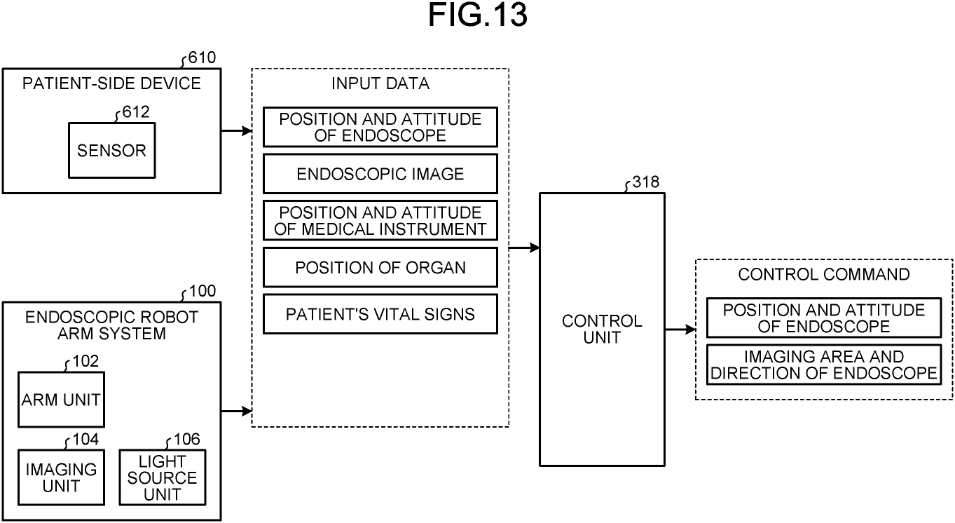

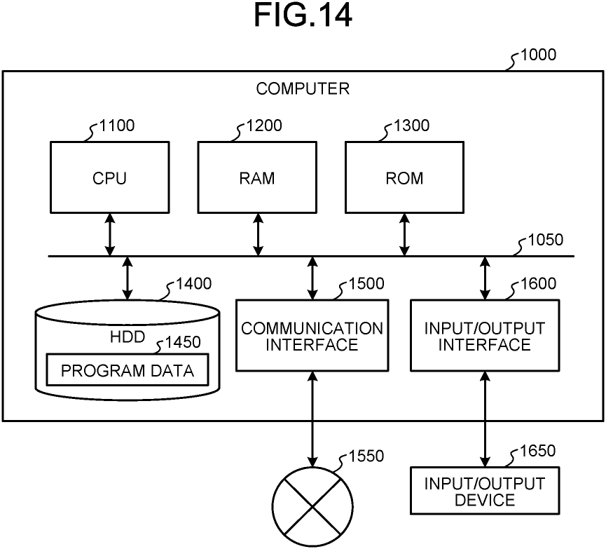

Next, a control method according to the present embodiment will be described with reference to FIGS. 12 and 13. FIG. 12 is a flowchart illustrating an example of a control method according to the present embodiment, and FIG. 13 is an explanatory diagram illustrating the control method according to the present embodiment. Specifically, as illustrated in FIG. 12, the control method according to the present embodiment can include a plurality of steps from Step S301 to Step S303. Details of each of these steps will be described below.

The control device 300 acquires various types of data regarding the state and the like of the endoscopic robot arm system 100 in real time from the endoscopic robot arm system 100 and the surgeon-side device 600 including the sensor 612 and the UI 602 (Step S301). The control device 300 calculates and outputs a control command based on the data acquired in Step S301 (Step S302). Next, the control device 300 controls the endoscopic robot arm system 100 based on the control command output in Step S302 (Step S303).

As described above, in the control method according to the present embodiment, the endoscopic robot arm system 100 can be controlled using only the reinforced autonomous movement control model.

### 5. Summary

As described above, in the embodiment of the present disclosure, the movement of the scope work in the clinical field and data of a resulting state obtained are input to the learning device as the input data and the training data, and the supervised learning is performed to generate the learning model for the autonomous scope work. Next, in the present embodiment, the data regarding the movement of the scope work (input data) and evaluation data for the data are input to the learning device as the training data, and the supervised learning is performed, thereby generating the learning model for outputting the “reward” given to the appropriate scope work. Furthermore, in the present embodiment, the reinforcement learning is performed using the learning model for autonomous scope work and the learning model for outputting the “reward”. In other words, in the present embodiment, by combining the supervised learning and the reinforcement learning, it is possible to efficiently acquire the learning model for autonomously performing the scope work in consideration of human sensitivity while covering a wider range of situations even in a case where only a small amount of clinical data can be obtained.

Note that the learning model related to the “reward” according to the embodiment of the present disclosure can also be applied to a test for certifying a skill of the scopist or assessment in the endoscopic surgery. Furthermore, the embodiment of the present disclosure is not limited to application to the scope work, and for example, can also be applied to a case where a part of movement (task) in the surgery is autonomously executed, such as suturing the surgical site with the medical instrument supported by the arm unit 102.

### 6. Hardware Configuration

An information processing apparatus such as the learning device 200 according to the embodiment described above is realized by, for example, a computer 1000 having a configuration as illustrated in FIG. 14. Hereinafter, the learning device 200 according to the embodiment of the present disclosure will be described as an example. FIG. 14 is a hardware configuration diagram illustrating an example of a computer that implements the learning device 200 according to the embodiment of the present disclosure. The computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates based on a program stored in the ROM 1300 or the HDD 1400, and controls each unit. For example, the CPU 1100 develops a program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a computer-readable recording medium that non-transiently records a program executed by the CPU 1100, data used by the program, and the like. Specifically, the HDD 1400 is a recording medium that records a program for the medical arm control method, which is an example of the program data 1450, according to the present disclosure.

The communication interface 1500 is an interface for the computer 1000 to connect to an external network 1550 (e.g., the Internet). For example, the CPU 1100 receives data from another apparatus or transmits data generated by the CPU 1100 to another apparatus via the communication interface 1500.

The input/output interface 1600 is an interface for connecting an input/output device 1650 and the computer 1000. For example, the CPU 1100 receives data from an input device such as a keyboard or a mouse via the input/output interface 1600. In addition, the CPU 1100 transmits data to an output device such as a display, a speaker, or a printer via the input/output interface 1600. Furthermore, the input/output interface 1600 may function as a media interface that reads a program or the like recorded on a predetermined computer-readable recording medium (medium). The medium is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, when the computer 1000 functions as the learning device 200 according to the embodiment of the present disclosure, the CPU 1100 of the computer 1000 executes a program for generating a model loaded on the RAM 1200. In addition, the HDD 1400 may store a program for generating the model according to the embodiment of the present disclosure. Note that the CPU 1100 reads the program data 1450 from the HDD 1400 and executes the program data 1450. However, as another example, an information processing program may be acquired from another device via the external network 1550.

Furthermore, the learning device 200 according to the present embodiment may be applied to a system including a plurality of devices on the premise of connection to a network (or communication between devices), such as cloud computing.

An example of the hardware configuration of the learning device 200 has been described above. Each of the above-described components may be configured using a general-purpose member, or may be configured by hardware specialized for the function of each component. This configuration can be appropriately changed according to a technical level at the time of implementation.

### 7. Supplement

Note that the embodiment of the present disclosure described above can include, for example, the control method executed by the control device or the control system as described above, the program for causing the control device to function, and the non-transitory tangible medium in which the program is recorded. Further, the program may be distributed via a communication line (including wireless communication) such as the Internet.

Furthermore, each step in the control method of the embodiment of the present disclosure described above may not necessarily be processed in the described order. For example, each step may be implemented in an appropriately changed order. In addition, each step may be partially implemented in parallel or individually instead of being implemented in time series. Furthermore, the process in each step does not necessarily have to be performed according to the described method, and may be performed, for example, by another method by another functional unit.

Among the processes described in the above embodiments, all or a part of the processes described as being automatically performed can be manually performed, or all or a part of the processes described as being manually performed can be automatically performed by a known method. In addition, the processing procedure, specific name, and information including various data and parameters illustrated in the above document and the drawings can be arbitrarily changed unless otherwise specified. For example, various types of information illustrated in each drawing are not limited to the illustrated information.

In addition, each component of each device illustrated in the drawings is functionally conceptual, and is not necessarily physically configured as illustrated in the drawings. In other words, a specific form of distribution and integration of each device is not limited to the illustrated form, and all or a part thereof can be functionally or physically distributed and integrated in an arbitrary unit according to various loads, usage conditions, and the like.

Although the preferred embodiment of the present disclosure has been described in detail with reference to the accompanying drawings, the technical scope of the present disclosure is not limited to these examples. It is obvious that a person having ordinary knowledge in the technical field of the present disclosure can conceive various changes or modifications within the scope of the technical idea described in the claims, and it is naturally understood that these also belong to the technical scope of the present disclosure.

Furthermore, the effects described in the present specification are merely illustrative or exemplary, and are not restrictive. In other words, the technology according to the present disclosure can exhibit other effects obvious to those skilled in the art from the description of the present specification in addition to or instead of the above effects.

The present technology can also have the following configurations.

A medical arm control system comprising:
a first determination unit that performs supervised learning using first input data and first training data, and generates an autonomous movement control model for autonomously moving a medical arm;
a second determination unit that performs supervised learning using second input data and second training data, and generates a reward model for calculating a reward to be given to a movement of the medical arm; and
a reinforcement learning unit that executes the reward model using third input data, and reinforces the autonomous movement control model using the reward calculated by the reward model.

The medical arm control system according to (1), wherein the medical arm supports a medical observation device.

The medical arm control system according to (2), wherein the medical observation device is an endoscope.

The medical arm control system according to (1), wherein the medical arm supports a medical instrument.

The medical arm control system according to any one of (1) to (3), wherein the first input data includes information regarding at least one of a position and an attitude of the medical arm, a position and an attitude of a medical instrument, surgical site information, patient information, and an image.

The medical arm control system according to (5), wherein the first input data and the first training data are clinical data, simulated clinical data, or virtual clinical data.

The medical arm control system according to (5) or (6), wherein the first training data includes information regarding at least one of the position and the attitude of the medical arm, and image information.

The medical arm control system according to (7), wherein the autonomous movement control model outputs information regarding at least one of the position, the attitude, a speed, and an acceleration of the medical arm and an imaging condition of the image.

The medical arm control system according to any one of (5) to (8), wherein the second input data includes at least one of the patient information and the image.

The medical arm control system according to (9), wherein the second input data is clinical data, simulated clinical data, or virtual clinical data.

The medical arm control system according to any one of (5) to (10), wherein the patient information includes information regarding at least one of a heart rate, a pulse, a blood pressure, a blood flow oxygen concentration, brain waves, respiration, sweating, myoelectric potential, a skin temperature, and a skin electrical resistance of a patient.

The medical arm control system according to any one of (5) to (11), wherein the surgical site information includes information regarding at least one of a type, a position, and an attitude of an organ, and a positional relationship between the medical instrument and the organ.

The medical arm control system according to any one of (1) to (12), further comprising a control unit that controls the medical arm according to the reinforced autonomous movement control model.

The medical arm control system according to any one of (1) to (13), wherein the second training data includes an evaluation score of a state of the medical arm.

The medical arm control system according to (14), wherein the evaluation score is a subjective evaluation score by a doctor.

The medical arm control system according to any one of (1) to (15), wherein the third input data is virtual clinical data.

A medical arm device which stores an autonomous movement control model obtained by reinforcing a control model for autonomously moving a medical arm using a reward obtained by inputting third input data to a reward model for calculating the reward to be given to a movement of the medical arm, the control model being generated by performing supervised learning using first input data and first training data, the reward model being generated by performing supervised learning using second input data and second training data.

A medical arm control method, by a medical arm control system, comprising:
reinforcing an autonomous movement control model for autonomously moving the medical arm
using a reward obtained by inputting third input data to a reward model for calculating the reward to be given to a movement of the medical arm, the autonomous movement control model being generated by performing supervised learning using first input data and first training data, the reward model being generated by performing supervised learning using second input data and second training data; and
controlling the medical arm using the reinforced autonomous movement control model.

A program causing a computer to function as:
a first determination unit that performs supervised learning using first input data and first training data, and generates an autonomous movement control model for autonomously moving a medical arm;
a second determination unit that performs supervised learning using second input data and second training data, and generates a reward model for calculating a reward to be given to a movement of the medical arm; and
a reinforcement learning unit that executes the reward model using third input data, and reinforces the autonomous movement control model using the reward calculated by the reward model.

Reference Signs List

| | |
|---|---|
| 10 | MEDICAL OBSERVATION SYSTEM |
| 100 | ENDOSCOPIC ROBOT ARM SYSTEM |
| 102 | ARM UNIT |
| 104 | IMAGING UNIT |
| 106 | LIGHT SOURCE UNIT |
| 200 | LEARNING DEVICE |
| 210, 312 | INFORMATION ACQUISITION UNIT |
| 222, 224 | MACHINE LEARNING UNIT |
| 230 | REINFORCEMENT LEARNING UNIT |
| 232 | SIMULATOR UNIT |
| 234 | EVALUATION UNIT |
| 236 | UPDATE UNIT |
| 240, 340 | STORAGE UNIT |
| 250, 320 | OUTPUT UNIT |
| 300 | CONTROL DEVICE |
| 310 | PROCESSING UNIT |
| 314 | IMAGE PROCESSING UNIT |
| 316 | MODEL ACQUISITION UNIT |
| 318 | CONTROL UNIT |
| 500 | PRESENTATION DEVICE |
| 600 | SURGEON-SIDE DEVICE |
| 602 | UI |
| 610 | PATIENT-SIDE DEVICE |
| 612 | SENSOR |

1] A medical arm control system comprising:
a first determination unit that performs supervised learning using first input data and first training data, and generates an autonomous movement control model for autonomously moving a medical arm;
a second determination unit that performs supervised learning using second input data and second training data, and generates a reward model for calculating a reward to be given to a movement of the medical arm; and
a reinforcement learning unit that executes the reward model using third input data, and reinforces the autonomous movement control model using the reward calculated by the reward model.

2] The medical arm control system according to claim 1, wherein the medical arm supports a medical observation device.

3] The medical arm control system according to claim 2, wherein the medical observation device is an endoscope.

4] The medical arm control system according to claim 1, wherein the medical arm supports a medical instrument.

5] The medical arm control system according to claim 1, wherein the first input data includes information regarding at least one of a position and an attitude of the medical arm, a position and an attitude of a medical instrument, surgical site information, patient information, and an image.

6] The medical arm control system according to claim 5, wherein the first input data and the first training data are clinical data, simulated clinical data, or virtual clinical data.

7] The medical arm control system according to claim 5, wherein the first training data includes information regarding at least one of the position and the attitude of the medical arm, and image information.

8] The medical arm control system according to claim 7, wherein the autonomous movement control model outputs information regarding at least one of the position, the attitude, a speed, and an acceleration of the medical arm and an imaging condition of the image.

9] The medical arm control system according to claim 5, wherein the second input data includes at least one of the patient information and the image.

10] The medical arm control system according to claim 9, wherein the second input data is clinical data, simulated clinical data, or virtual clinical data.

11] The medical arm control system according to claim 5, wherein the patient information includes information regarding at least one of a heart rate, a pulse, a blood pressure, a blood flow oxygen concentration, brain waves, respiration, sweating, myoelectric potential, a skin temperature, and a skin electrical resistance of a patient.

12] The medical arm control system according to claim 5, wherein the surgical site information includes information regarding at least one of a type, a position, and an attitude of an organ, and a positional relationship between the medical instrument and the organ.

13] The medical arm control system according to claim 1, further comprising a control unit that controls the medical arm according to the reinforced autonomous movement control model.

14] The medical arm control system according to claim 1, wherein the second training data includes an evaluation score of a state of the medical arm.

15] The medical arm control system according to claim 14, wherein the evaluation score is a subjective evaluation score by a doctor.

16] The medical arm control system according to claim 1, wherein the third input data is virtual clinical data.

17] A medical arm device which stores an autonomous movement control model obtained by reinforcing a control model for autonomously moving a medical arm using a reward obtained by inputting third input data to a reward model for calculating the reward to be given to a movement of the medical arm, the control model being generated by performing supervised learning using first input data and first training data, the reward model being generated by performing supervised learning using second input data and second training data.

18] A medical arm control method, by a medical arm control system, comprising:
reinforcing an autonomous movement control model for autonomously moving the medical arm
using a reward obtained by inputting third input data to a reward model for calculating the reward to be given to a movement of the medical arm, the autonomous movement control model being generated by performing supervised learning using first input data and first training data, the reward model being generated by performing supervised learning using second input data and second training data; and
controlling the medical arm using the reinforced autonomous movement control model.

19] A program causing a computer to function as:
a first determination unit that performs supervised learning using first input data and first training data, and generates an autonomous movement control model for autonomously moving a medical arm;
a second determination unit that performs supervised learning using second input data and second training data, and generates a reward model for calculating a reward to be given to a movement of the medical arm; and
a reinforcement learning unit that executes the reward model using third input data, and reinforces the autonomous movement control model using the reward calculated by the reward model.
